SEQUENCE LISTING (26 sequences)

SEQ ID NO: 1 - protein, 392 aa, Mycobacterium bovis
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 2 - protein, 71 aa, Mycobacterium bovis
ASTRQALRPRADGPVGAAAEQVGGQSQLVSAQGSQGMGGPVGMGGMHPSSGASKGTTTKKYSEGAAAGTED

SEQ ID NO: 3 - protein, 60 aa, Mycobacterium bovis
SLEYFEKALEELAAAFPGDGWLGSAADKYAGKNRNHVNFFQELADLDRQLISLIHDQANA

SEQ ID NO: 4 - protein, 34 aa, Mycobacterium bovis
MSRAFIIDPTISAIDGLYDLLGIGIPNQGGILYS

SEQ ID NO: 5 - protein, 298 aa, Mycobacterium bovis
VQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 6 - protein, 193 aa, Mycobacterium bovis
MKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 7 - protein, 220 aa, Mycobacterium bovis
MINVQAKPAAAASLAAIAIAFLAGCSSTKPVSQDTSPKPATSPAAPVTTAAMADPAADLIGRGCAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVCGGVHTANATVYMIDTVLMPPAQ

SEQ ID NO: 8 - protein, 94 aa, Mycobacterium bovis
TEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 9 - protein, 99 aa, Mycobacterium bovis
AEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 10 - protein, 157 aa, Mycobacterium bovis
MNNLALWSRPVWDVEPWDRWLRDFFGPAATTDWYRPVAGDFTPAAEIVKDGDDAVVRLELPGIDVDKNVELDPGQPVSRLVIRGEHRDEHTQDAGDKDGRTLREIRYGSFRRSFRLPAHVTSEAIAASYDAGVLTVRVAGAYKAPAETQAQRIAITK

SEQ ID NO: 11 - protein, 99 aa, Mycobacterium bovis
MEKMSHDPIAADIGTQVSDNALHGVTAGSTALTSVTGLVPAGADEVSAQAATAFTSEGIQLLASNASAQDQLHRAGEAVQDVARTYSQIDDGAAGVFAE

SEQ ID NO: 12 - protein, 368 aa, Mycobacterium bovis
MLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLLRAESLPGAGGSLTRTPLMSQLIEKPVAPSVMPAAAAGSSATGGAAPVGAGAMGQGAQSGGSTRPGLVAPAPLAQEREEDDEDDWDEEDDW

SEQ ID NO: 13 - protein, 136 aa, Mycobacterium bovis
MTTTPARFNHLVTVTDLETGDRAVCDRDQVAETIRAWFPDAPLEVREALVRLQAALNRHEHTGELEAFLRISVEHADAAGGDECGPAILAGRSGPEQAAINRQLGLAGDDEPDGDDTPPWSRMIGLGGGSPAEDER

SEQ ID NO: 14 - protein, 90 aa, Mycobacterium bovis
MAETPDHAELRRRIADMAFNADVGMATCKRCGDAVPYIILPNLQTGEPVMGVADNKWKRANCPVDVGKPCPFLIAEGVADSTDDTIEVDQ

SEQ ID NO: 15 - protein, 171 aa, Mycobacterium bovis
MSRHHNIVIVCDHGRKGDGRIEHERCDLVAPIIWVDETQGWLPQAPAVATLLDDDNQPRAVIGLPPNESRLRPEMRRDGWVRLHWEFACLRYGAAGVRTCEQRPVRVRNGDLQTLCENVPRLLTGLAGNPDYAPGFAVQSDAVVVAMWLWRTLCESDTPNKLRATPTRGSC

SEQ ID NO: 16 - protein, 16 aa, Mycobacterium bovis
KWDATATELNNALQNL

SEQ ID NO: 17 - protein, 23 aa, Artificial
  chemically synthesized
KGSGSNIRQAGVQYSRADEEQQQ

SEQ ID NO: 18 - protein, 22 aa, Artificial
  chemically synthesized
KGSGSMAEMKTDAATLAQEAGN

SEQ ID NO: 19 - protein, 25 aa, Artificial
  chemically synthesized
KGSGSSVQGMSQDPVAVAASNNPEL

SEQ ID NO: 20 - protein, 28 aa, Artificial
  chemically synthesized
KEKDKDGNGLVCGGVHTANATVYMIDTV

SEQ ID NO: 21 - protein, 18 aa, Artificial
  chemically synthesized
DKGDGSAPKTYCEELKGT

SEQ ID NO: 22 - protein, 17 aa, Mycobacterium bovis
GVADNKWKRANCPVDVG

SEQ ID NO: 23 - protein, 25 aa, Artificial
  chemically synthesized
KGSGSSVQGMSQDPVAVAASNNPEL

SEQ ID NO: 24 - protein, 22 aa, Artificial
  chemically synthesized
KGSGSMAEMKTDAATLAQEAGN

SEQ ID NO: 25 - protein, 23 aa, Artificial
  chemically synthesized
KGSGSNIRQAGVQYSRADEEQQQ

SEQ ID NO: 26 - protein, 18 aa, Mycobacterium bovis
VVRFQEAANKQKQELDEI

(19) United States
(12) Patent Application Publication
(10) Pub. No.: US 2011/0020404 A1
(43) Pub. Date: Jan. 27, 2011

(54) DIAGNOSTIC METHOD AND KIT

(75) Inventors: Shane A. Olwill, Belfast (GB); James A. Johnston, Belfast (GB); Hang Fai Kwok, Belfast (GB); Clare Whelan, Tipperary (IE); John Clarke, Tipperary (IE)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignees: FUSION ANTIBODIES LIMITED, Belfast (GB); ENFER TECHNOLOGY LIMITED, Tipperary (IE)

(21) Appl. No.: 12/673,798

(22) PCT Filed: Aug. 18, 2008
(86) PCT No.: PCT/GB08/50721

§ 371 Date: Jul. 2, 2010

(30) Foreign Application Priority Data
Aug. 17, 2007 (GB) 0716070.8
Aug. 15, 2008 (IE) S2008/0670

(51) Int. Cl. A61K 39/04; C12Q 1/04; C12Q 1/02; G01N 33/53; C12Q 1/68; A61P 31/06

(52) U.S. Cl. 424/248.1; 435/34; 435/29; 435/7.1; 435/6

(57) ABSTRACT

The invention relates to methods for diagnosis of tuberculosis in an animal, the methods comprising determining the presence or absence of, or an immune response to a group of antigens comprising (i) the group consisting of Rv3616 antigen, MPB70 antigen, MPB83 antigen, and ESAT 6 antigen; or (ii) at least seven of the antigens of the group consisting of Rv3616 antigen, MPB70 antigen, MPB83 antigen, ESAT 6 antigen, CFP10 antigen, α crystalline 2 antigen, PE35 antigen, and PPE68 antigen. Also provided is a method for the diagnosis of the presence of tuberculosis in a reservoir animal e.g. a badger, said method comprising determining the presence or absence of, or an immune response to Rv3616 antigen, MPB70 antigen, and MPB83 antigen. Kits for carrying out the methods are also provided.

### FIELD OF THE INVENTION

The invention relates to methods and kits for the diagnosis and/or herd profiling of Mycobacterium infected or contaminated animals.

### BACKGROUND TO THE INVENTION

Tuberculosis (TB), one of the most widespread infectious diseases, is the leading cause of death due to a single infectious agent among adults in the world. Mycobacterium tuberculosis is the most common cause of human TB.

However, an unknown proportion of cases of zoonotic tuberculosis are due to M. bovis with some due to Mycobacterium avium. Thus, infection of the animal population not only places a strain on economic resources but also presents a threat to human health. The requirement for successful diagnostic assays and potential vaccines has increased due to the recent rise in TB levels in the cattle population of countries such as Great Britain and the wild life reservoirs of the world.

Great Britain performs some 4.6 million tests on bovine TB, costing the tax payer £88 m per annum (TB Conference M. bovis IV, Dublin 2005). Bovine TB varies regionally within GB, with the worst incidence seen in South Wales, Cornwall and Gloucestershire where 25% of all animals are infected. The incidence of TB is increasing at a rate of 2.5% per year in previously uninfected herds (TB Conference M. bovis IV, Dublin 2005).

In order to monitor and control the disease herd profiling is necessary. However, the methods currently used to monitor tuberculosis in animals suffer from a number of drawbacks. Nowadays, the disease control programmes for bovine TB carried out in most countries (e.g. US, Australia and GB etc.) are based on a test and removal strategy utilizing the intradermal skin test, which relies on PPD, a purified protein derivative of M. bovis strain AN5, to elicit an immune response in infected cattle.

In cattle, the intradermal skin tests currently used are the Caudal-fold Tuberculin Test (CFT) and the Comparative cervical tuberculin test (CCT). The Caudal-fold Tuberculin Test (CFT) is the primary screening test used to identify cattle herds potentially infected with bovine tuberculosis. It measures the immune response to Purified Protein Derivative (PPD) tuberculin (M. bovis AN5). If the animal's immune system recognizes the PPD, inflammatory cells (white blood cells) migrate to the injected site to help get rid of the foreign material (PPD). This cell mediated immune response may be recognized by swelling or discoloration at the site where PPD was injected. However, in 5% of cases, the CFT test may result in false-positive test results (due to exposure to or infection with other closely related bacteria, such as M. avium and M. paratuberculosis) or, in 15% of cases, in false-negative test results—where a very early stage of infection with bovine TB is not detected. As a follow up test to the CFT, the Comparative cervical tuberculin test (CCT) may be performed. The CCT test, in which suspect animals are injected with PPD avian and PPD bovine in the cervical (neck) region, is a more definitive test designed to determine if a response noted on the CFT test is more likely due to infection with M. avium or M. bovis. CCT Test—suspect cattle are subjected to further testing using necropsy and further diagnostic testing.

Disease control based on the skin test can be complemented by the gamma-interferon test, which measures the animals T cell response when exposed to PPD material. The gamma-interferon test is utilised as a second line diagnostic for ‘skin test positive’ animals (reports suggest it is more sensitive, but sometimes less specific than a skin test). It detects the cell-mediated immune response that develops following M. bovis infection (2 weeks).

The Gamma-Interferon test is not used as a primary test for mass screening on its own because it does not detect all skin test-positive infected animals; it is relatively expensive and is less specific than the CCT.

Due to their modes of action, the specificity of both the intradermal skin test and the gamma interferon test will always be an issue. The cross reactions induced by different mycobacteria strains and environmental mycobacteria such as M. microti and M. africanum and the conflicting requirements between specificity and sensitivity of the test antigens, all accrue to the difficulties in establishing a satisfactory serological protocol for bovine TB.

One of the problems associated with the complex antigenic nature of mycobacteria is the definition of those proteins which are important targets of the immune system and are thus likely present in large numbers of field samples. It is also important to recognize that there is regional variation in the infectious M. bovis strains.

An antibody assay developed using strain specific proteins could resolve both specificity and sensitivity issues.

A number of methods of discriminating between strains of tuberculosis have been suggested. U.S. Pat. No. 6,686,166B, WO 2004/083448A, US2004/0063923A, and U.S. Pat. No. 6,291,190B describe 129 genetic marker sequences which are suggested for use in the identification of strains of mycobacteria. However, as described herein the mere identification of such markers does not equate with practical utility in a diagnostic method. Many markers are not expressed sufficiently to reliably be used in the identification of a strain. Further, as described further below, the amount of expression of individual markers varies considerably, not only between strains, but within strains geographically and within strains dependent on the stage of infection.

US2005/0272104A suggests the use of the PPD antigens ESAT-6 and CFP-10 in the detection of Mycobacterium tuberculosis in humans. In general however, antibody (Ab) tests based upon PPD tuberculins are characterised by a low discriminating power, with the distribution of the antibody titers between infected and non-infected animals being widely overlapping (Amadori et al. 1998).

U.S. Pat. No. 7,192,721 describes a test kit for detection of TB in a different non-primate mammals, the kit comprising a mycobacterial antigen cocktail comprising ESAT-6/CFP10 and 16 kDa/MPB83 fusion proteins. In a sample of 85 cattle, the sensitivity demonstrated was 79%; however, in 513 cattle, the sensitivity was 73%.

Due to the heterogeneity of the disease, no single antigen is present in all cases of infection. However, although a great many antigens associated with tuberculosis infection have been identified in the prior art, to date, the use of particular combinations has not resulted in a test with an acceptable level of sensitivity and specificity to avoid the many false positive and false negative test results associated with conventional TB testing.

However, although relatively high sensitivity has been reported for some prior art tuberculosis kits, the sensitivity may only be maintained across selected populations of animals, for example within particular herds or in defined geographical regions, where variation in prevalent strains of tuberculosis is limited and thus variation between animals in their response to infection with such strains is limited. Presently available diagnostic kits do not provide specific combinations of antigens with sufficient coverage over different populations, herds and geographical areas for the reliable diagnosis of tuberculosis.

Although many antigens have been identified, by simply increasing the number of antigens in a test kit, the sensitivity and specificity of the test may not necessarily be improved. It is recognised in the art that, where diagnostic kits comprise too many antigenic additional antigens, the specificity and sensitivity of the kit may in fact be reduced. For example, US 792,721 describes a decrease in sensitivity and specificity associated with increasing the number of antigens used in the kits described therein. Thus, crucial to the design of an effective kit is the selection of antigens to be used therein.

As well as the problems discussed above with respect to identification of antigens for reliable diagnosis of tuberculosis infection, the formats of many diagnostic kits currently used may contribute to the lack of reliability. Cumulative results from animals experimentally infected with Mbv have shown there may be a transient antibody response to some antigens that occur at different stages of infection, further complicating development of a diagnostic assay (Amadori, et al 2002. Vet. Microbiol. 85:379-389). The advances that have been made show that some of the difficulties in developing an optimal assay can be overcome by including multiple antigens in the assay (Aagaard, et al 2006. J. Clin. Microbiol. 44:4326-4335., Amadori, et al 2002. Vet. Microbiol. 85:379-389). Although some success has also been made in identifying antigens potentially useful in multiplexed diagnostic assays (Aagaard, et al 2006. J. Clin. Microbiol. 44:4326-4335., Koo et al 2005. J. Clin. Microbiol. 43:4498-4506, Lightbody, et al 2000. Vet. Microbiol. 75:177-188, McNair et al 2001. Scand. J. Immunol. 53:365-371., Pollock, J. M. and P. Andersen. 1997. Infect. Immun. 65:2587-2592, Pollock, J. M. and P. Andersen. 1997. J. Infect. Dis. 175:1251-1254, Pollock, et al 2000. Vet. Rec. 146:659-665, Wiker et al 1998. Infect. Immun. 66:1445-1452). the necessity of using multiple antigens in an assay, has, however, introduced another challenge. Evaluation of the standard type of ELISA has shown sensitivity and specificity are reduced when multiple antigens are combined for analysis in a single well thus limiting the way a conventional ELISA can be used (Lyashchenko, et al 2000. J. Immunol. Methods 242:91-100). Fluorescence polarization has been evaluated as an alternative platform (Lin et al 1996. Clin. Diag. Lab. Immunol. 3:438-443, Surujballi et al 2002. Vet. Microbiol. 87:149-157). It has been successfully used in testing for Mbv in cattle, elk, llamas, and bison with a single antigen. A further improvement over an ELISA-based system has been the use of lateral flow technology (Koo et al 2005. J. Clin. Microbiol. 43:4498-4506, Lyashchenko, et al 2000. J. Immunol. Methods 242:91-100). This methodology has been used successfully for multiplexing antigens for visual analysis of patterns of antibody activity at the single animal level (Greenwald, et al 2003. Diagn. Microbiol. Infect. Dis. 46:197-203, Lyashchenko, et al 2006. Clin. Vaccine Immunol. 13:722-732, Waters, et al 2006. Clin. Vaccine Immunol. 13:648-654, Waters, et al 2006. Clin. Vaccine Immunol. 13:611-619, Wernery et al 2007. Vet. Microbiol. 122:108-115.). Other areas that have not been fully explored are electrochemiluminescence (Lu, Y. et al 2006. J. Immunol. Methods 314:74-79, Yan, et al. 2004. J. Immunol. Methods 288:47-54) and chemiluminescence Liew et al 2007. Biotechniques 42:327-333). Commercial imaging systems using these technologies have been developed for multi-plexing and analysis of antibody activity to multiple antigens (Liew et al 2007. Biotechniques 42:327-333, Lu, Y. et al 2006. J. Immunol. Methods 314:74-79, Yan, et al. 2004. J. Immunol. Methods 288:47-54). The platforms are designed for rapid processing and analysis of large numbers of samples. However, a number of problems persist with many of these methods.

A test which can accurately and reliably diagnose the presence of tuberculosis infection with satisfactory sensitivity and specificity across different populations of animals has eluded the field.

### SUMMARY OF THE INVENTION

As described herein, the present inventors have overcome a number of problems with the prior art diagnostic methods and have identified a number of polypeptides, which together are useful for the sensitive specific diagnosis and profiling of Mycobacterium.

Each of the specific polypeptides is capable of eliciting a strong immune response in the absence of adjuvant. The characterized proteins/fragments which are immunogenic (or early antigens) and specific to one or other mycobacterial strains can be used reliably for the diagnosis of tuberculosis infection. As described in the examples, such polypeptides elicit a strong immune response in serum from animals immunised with Mycobacterium Bovis. Significantly, the inventors have found that, when these polypeptides are used together in an assay, the sensitivity of tuberculosis testing can be vastly improved over the sensitivity obtained using prior art methods. Thus, the invention, for the first time, enables the use of a single test to reliably diagnose the presence or absence of tuberculosis infection in many different populations of animals across differing geographical areas and thus may form the basis of a universal test.

The invention relates to the use of specific polypeptides, antibodies, or nucleic acid molecules for the detection of TB disease or mycobacterial challenge. The use of a multi-peptide approach combined with the particular selection of highly immunogenic peptide fragments described herein facilitates the sensitive and specific diagnosis of TB.

Significantly, the specific combination of antigens used in the present invention allows diagnosis of tuberculosis infection across herds, geographical boundaries with levels of sensitivity specificity hitherto unobtainable across different populations of animals.

The polypeptides which may be used in the invention are SEQ ID NOs: 1-26 and variants and fragments thereof.

In one embodiment of the invention, the polypeptides which may be used in the invention comprise peptides having amino acid sequences comprising the amino acid sequences shown as one or more of SEQ ID NOs: 1-26.

corresponds to Rv3616C (full length)
SEQ ID NO: 1
MSRAFIIDPT ISAIDGLYDL LGIGIPNQGG ILYSSLEYFE
KALEELAAAF PGDGWLGSAA DKYAGKNRNH VNFFQELADL
DRQLISLIHD QANAVQTTRD ILEGAKKGLE FVRPVAVDLT
YIPVVGHALS AAFQAPFCAG AMAVVGGALA YLVVKTLINA
TQLLKLLAKL AELVAAAIAD IISDVADIIK GTLGEVWEFI
TNALNGLKEL WDKLTGWVTG LFSRGWSNLE SFFAGVPGLT
GATSGLSQVT GLFGAAGLSA SSGLAHADSL ASSASLPALA
GIGGGSGFGG LPSLAQVHAA STRQALRPRA DGPVGAAAEQ
VGGQSQLVSA QGSQGMGGPV GMGGMHPSSG ASKGTTTKKY
SEGAAAGTED AERAPVEADA GGGQKVLVRN VV
corresponds to residues 300-370 of rv3616c
(Mtb40):
SEQ ID NO: 2
ASTRQALRPRADGPVGAAAEQVGGQSQLVSAQGSQGMGGPVGMGGMHPSS
GASKGTTTKKYSEGAAAGTED
corresponds to residues 35-94 of Rv3616c:
SEQ ID NO: 3
SLEYFEKALEELAAAFPGDGWLGSAADKYAGKNRNHVNFFQELADLDRQL
ISLIHDQANA
corresponds to residues 1-34 of Rv3616c:
SEQ ID NO: 4
MSRAFIIDPTISAIDGLYDLLGIGIPNQGGILYS
corresponds to residues 95-392 of Rv3616c:
SEQ ID NO: 5
VQTTRDILEGAKKGLEFVRPVAVDLTYIPVVGHALSAAFQAPFCAGAMAV
VGGALAYLVVKTLINATQLLKLLAKLAELVAAAIADIISDVADIIKGTLG
EVWEFITNALNGLKELWDKLTGWVTGLFSRGWSNLESFFAGVPGLTGATS
GLSQVTGLFGAAGLSASSGLAHADSLASSASLPALAGIGGGSGFGGLPSL
AQVHAASTRQALRPRADGPVGAAAEQVGGQSQLVSAQGSQGMGGPVGMGG
MHPSSGASKGTTTKKYSEGAAAGTEDAERAPVEADAGGGQKVLVRNVV
corresponds to MPB70
SEQ ID NO: 6
MKVKNTIAATSFAAAGLAALAVAVSPPAAAGDLVGPGCAEYAAANPTGPA
SVQGMSQDPVAVAASNNPELTTLTAALSGQLNPQVNLVDTLNSGQYTVFA
PTNAAFSKLPASTIDELKTNSSLLTSILTYHVVAGQTSPANVVGTRQTLQ
GASVTVTGQGNSLKVGNADVVCGGVSTANATVYMIDSVLMPPA
corresponds to MPB83
SEQ ID NO: 7
MINVQAKPAAAASLAAIAIAFLAGCSSTKPVSQDTSPKPATSPAAPVTTA
AMADPAADLIGRGCAQYAAQNPTGPGSVAGMAQDPVATAASNNPMLSTLT
SALSGKLNPDVNLVDTLNGGEYTVFAPTNAAFDKLPAATIDQLKTDAKLL
SSILTYHVIAGQASPSRIDGTHQTLQGADLTVIGARDDLMVNNAGLVCGG
VHTANATVYMIDTVLMPPAQ
corresponds to Rv3875 (ESAT6):
SEQ ID NO: 8
TEQQWNFAGIEAAASAIQGNVTSIHSLLDEGKQSLTKLAAAWGGSGSEAY
QGVQQKWDATATELNNALQNLARTISEAGQAMASTEGNVTGMFA
corresponds to Rv3874 (CFP10):
SEQ ID NO: 9
AEMKTDAATLAQEAGNFERISGDLKTQIDQVESTAGSLQGQWRGAAGTAA
QAAVVRFQEAANKQKQELDEISTNIRQAGVQYSRADEEQQQALSSQMGF
corresponds to Rv0251c (α-crystallin 2):
SEQ ID NO: 10
MNNLALWSRPVWDVEPWDRWLRDFFGPAATTDWYRPVAGDFTPAAEIVKD
GDDAVVRLELPGIDVDKNVELDPGQPVSRLVIRGEHRDEHTQDAGDKDGR
TLREIRYGSFRRSFRLPAHVTSEAIAASYDAGVLTVRVAGAYKAPAETQA
QRIAITK
corresponds to PE35:
SEQ ID NO: 11
MEKMSHDPIAADIGTQVSDNALHGVTAGSTALTSVTGLVPAGADEVSAQA
ATAFTSEGIQLLASNASAQDQLHRAGEAVQDVARTYSQIDDGAAGVFAE
corresponds to PPE68
SEQ ID NO: 12
MLWHAMPPELNTARLMAGAGPAPMLAAAAGWQTLSAALDAQAVELTARLN
SLGEAWTGGGSDKALAAATPMVVWLQTASTQAKTRAMQATAQAAAYTQAM
ATTPSLPEIAANHITQAVLTATNFFGINTIPIALTEMDYFIRMWNQAALA
MEVYQAETAVNTLFEKLEPMASILDPGASQSTTNPIFGMPSPGSSTPVGQ
LPPAATQTLGQLGEMSGPMQQLTQPLQQVTSLFSQVGGTGGGNPADEEAA
QMGLLGTSPLSNHPLAGGSGPSAGAGLLRAESLPGAGGSLTRTPLMSQLI
EKPVAPSVMPAAAAGSSATGGAAPVGAGAMGQGAQSGGSTRPGLVAPAPL
AQEREEDDEDDWDEEDDW
corresponds to Rv1573:
SEQ ID NO: 13
MTTTPARFNHLVTVTDLETGDRAVCDRDQVAETIRAWFPDAPLEVREALV
RLQAALNRHEHTGELEAFLRISVEHADAAGGDECGPAILAGRSGPEQAAI
NRQLGLAGDDEPDGDDTPPWSRMIGLGGGSPAEDER
corresponds to Rv1580c:
SEQ ID NO: 14
MAETPDHAELRRRIADMAFNADVGMATCKRCGDAVPYIILPNLQTGEPVM
GVADNKWKRANCPVDVGKPCPFLIAEGVADSTDDTIEVDQ
corresponds to Rv1585c:
SEQ ID NO: 15
MSRHHNIVIVCDHGRKGDGRIEHERCDLVAPIIWVDETQGWLPQAPAVAT
LLDDDNQPRAVIGLPPNESRLRPEMRRDGWVRLHWEFACLRYGAAGVRTC
EQRPVRVRNGDLQTLCENVPRLLTGLAGNPDYAPGFAVQSDAVVVAMWLW
RTLCESDTPNKLRATPTRGSC
corresponds to Mb3905:
SEQ ID NO: 16
KWDATATELNNALQNL
corresponds to Mb3904(1):
SEQ ID NO: 17
KGSGSNIRQAGVQYSRADEEQQQ
corresponds to Mb3904(2):
SEQ ID NO: 18
KGSGSMAEMKTDAATLAQEAGN
corresponds to Mb2900:
SEQ ID NO: 19
KGSGSSVQGMSQDPVAVAASNNPEL
corresponds to Mb Mb2898:
SEQ ID NO: 20
KEKDKDGNGLVCGGVHTANATVYMIDTV
corresponds to Mb2002c:
SEQ ID NO: 21
DKGDGSAPKTYCEELKGT
corresponds to Mb1606c:
SEQ ID NO: 22
GVADNKWKRANCPVDVG
corresponds to PEP1 (MPB70):
SEQ ID NO: 23
KGSGSSVQGMSQDPVAVAASNNPEL
corresponds to PEP4 (CFP10)
SEQ ID NO: 24
KGSGSMAEMKTDAATLAQEAGN
corresponds to PEP9 (CFP10)
SEQ ID NO: 25
KGSGSNIRQAGVQYSRADEEQQQ
corresponds to PEP 17 (CFP10)
SEQ ID NO: 26
VVRFQEAANKQKQELDEI

Underlined sequences, as shown above for some peptides, were added to enhance the hydrophilicity of the peptides for binding to the surface. Where reference is made herein to use of such peptides, it should be understood that such peptides may be employed with or without the hydrophilicity enhancing sequence.

The inventors have found that the peptides having amino acid sequences SEQ ID NO: 1 to SEQ ID NO: 26 each elicit a strong immune response in serum from animals challenged with mycobacterial strains. Significantly, the inventors have found that by employing seven or more of the antigens, selected from the group consisting of an Rv3616 antigen, an MPB70 antigen, an MPB83 antigen, an ESAT 6 antigen, a CFP10 antigen, an α crystalline 2 antigen, a PE35 antigen, and a PPE68 antigen, the presence of tuberculosis may be identified in animals with a degree of sensitivity hitherto not obtainable.

Furthermore the inventors have also shown that a diagnostic test for tuberculosis infection demonstrating both high sensitivity and specificity may be based on only four antigens, namely an Rv3616 antigen, an MPB70 antigen, an MPB83 antigen, and an ESAT 6 antigen.

In one embodiment, the MPB70 antigen is the full length MPB70 protein, for example that having the amino acid sequence shown as Sequence ID No: 6. In another embodiment, the MPB70 antigen is a linear MPB70 epitope, for example the pep1 (MPB70) antigen having the amino acid sequence shown as Sequence ID No: 23. In a particular embodiment of the invention, the test is based on an Rv3616 antigen, an MPB70 antigen, an MPB83 antigen, and an ESAT 6 antigen and a PEP1 (MPB70) antigen. As described in the Examples, the sensitivity of the diagnostic test for diagnosing bovine tuberculosis increased from approximately 70% using a test based on determination of the presence of an immune response to Rv3616 antigen, an MPB70 antigen, an MPB83 antigen, and an ESAT 6 antigen to over 93% using a test based on the determination of the presence of an immune response to the same four antigens and to a pep1 (MPB70) antigen. The marked improvement obtained when determining the presence of a response to the full length antigen and a response to the linear epitope was particularly unexpected.

Accordingly, in a first aspect of the invention, there is provided a method of diagnosis of tuberculosis in an animal, the method comprising the steps:

providing a biological sample from said animal; and determining the presence or absence of a group of antigens, or the presence or absence of an immune response to each member of said group of antigens, wherein said group of antigens comprises
(i) the group consisting of an Rv3616 antigen, an MPB70 antigen, an MPB83 antigen, and an ESAT 6 antigen; or
(ii) at least seven of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen;
wherein the identification of the presence of two or more of said antigens of said group of antigens, or of an immune response thereto, is indicative of the presence of tuberculosis infection in the animal.

In one embodiment, the identification of the presence of one or more of said antigens of said group of antigens, or of an immune response thereto, is indicative of the presence of tuberculosis infection in the animal.

The peptides having amino acid sequences SEQ ID NO: 1 to SEQ ID NO: 26 each elicit strong immune responses in serum from animals infected with Mycobacterium bovis but are not present in either environmental strains of Mycobacterium or avian PPD strains.

In a second aspect, the present invention provides an assay for the detection of the presence of Mycobacterium, for example Mycobacterium bovis, in a biological sample, said method comprising the steps:
providing a biological sample; and determining the presence or absence of, or an immune response to, each member of a group of antigens in the biological sample, wherein said group of antigens comprises
   (i) the group consisting of an Rv3616 antigen, an MPB70 antigen, an MPB83 antigen, and an ESAT 6 antigen; or
(ii) at least seven of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen; in the biological sample;
wherein the identification of the presence of two or more of said antigens or of an immune response thereto is indicative of the presence of Mycobacterium in the biological sample.

In one embodiment, the identification of the presence of one or more of said antigens or of an immune response thereto is indicative of the presence of Mycobacterium in the biological sample.

In one embodiment of the first or second aspect of the invention, the method involves determining the presence or absence of a group of antigens, or the presence or absence of an immune response to each member of said group of antigens, wherein said group of antigens comprises the group consisting of an Rv3616 antigen, an MPB70 antigen, an MPB83 antigen, and an ESAT 6 antigen, wherein the MPB70 antigen is a full length MPB70 protein. In another embodiment of the first or second aspects of the invention, the method involves determining the presence or absence of a group of antigens, or the presence or absence of an immune response to each member of said group of antigens, wherein said group of antigens comprises the group consisting of an Rv3616 antigen, an MPB70 antigen, an MPB83 antigen, and an ESAT 6 antigen, wherein the MPB70 antigen is an MPB70 linear epitope, for example PEP1 (MPB70). In a particular embodiment of the first and second aspects of the invention, the method involves determining the presence or absence of a group of antigens, or the presence or absence of an immune response to each member of said group of antigens, wherein said group of antigens comprises both a full length MPB70 protein and an MPB70 linear epitope, for example PEP1 (MPB70).

In particular embodiments of the invention, the presence or absence of each of the antigens of the group consisting of Rv3616 antigen, an MPB70 antigen, and MPB83 antigen, an ESAT 6 antigen, a CFP10 antigen, an α crystalline 2 antigen, a PE35 antigen, and a PPE68 antigen is determined. As described in the examples, the use of these antigens greatly enhances the sensitivity of the test in identifying tuberculosis, providing unprecedented coverage across populations.

Unless the context demands otherwise (for example where reference is made to an MPB70 antigen and a pep1 (MPB70) antigen within the same context), reference to an MPB70 antigen should be understood to encompass an MPB70 antigen having the sequence shown as Sequence ID No: 6 or a pep1 (MPB70) antigen having the sequence shown as Sequence ID No: 23, or both. In one embodiment of the first and second aspects of the invention, the presence or absence of an immune response to both an MPB70 antigen having the sequence shown as Sequence ID No: 6 and a pep1 (MPB70) antigen having the sequence shown as Sequence ID No: 23 are determined. The combination of use of the MPB70 peptide together with the recombinant protein gives full coverage as this allows the identification of antibodies to both linear and conformational epitopes.

Likewise, unless the context demand otherwise, where reference is made to a CFP10 antigen, such reference should be understood to encompass one or more of a CFP10 antigen having the sequence shown as Sequence ID No: 9, a pep4 (CFP10) antigen having the sequence shown as Sequence ID No: 24, a pep9 (CFP10) antigen having the sequence shown as Sequence ID No: 25, or a pep17 (CFP10) antigen having the sequence shown as Sequence ID No: 26.

The present inventors have established that, even if an animal has been exposed to bovine PPD, the detectable immune response to the PPD test, in many cases, decreases rapidly after the initial exposure. Thus, although exposure to one or more of these antigens may be detectable in an animal uninfected with tuberculosis but which has been tested with bovine PPD within 2 weeks of the PPD test, within one month or so, the antigenic response will, in many cases, be undetectable. Thus, in general, unless a particular herd has been tested with PPD within one month preceding testing the animals with the method or kit of the present invention, the identification of the presence of an immune response to one or more of these antigens should be indicative of infection with Mycobacterium bovis and not merely exposure to bovine PPD.

However, the antigens having Sequence ID No: 9, Sequence ID No: 10 and Sequence ID No: 11 (rv1573, Rv1580c and Rv1585c) are not expressed in bovine PPD and thus may be used to confirm that the presence of a positive result is indicative of infection with bovine tuberculosis and not merely past exposure to PPD via e.g. the intradermal skin test.

Accordingly, in one embodiment of the first and second aspects of the invention, the method further includes determining the presence or absence of at least one of the antigens, or an immune response to at least one of the antigens, of the group consisting of an Rv1573 antigen, an Rv1580c antigen and an Rv1585c antigen. The identification of the presence of one or more of the Rv1573 antigen, the Rv1580c antigen and the Rv1585c antigen or of an immune response thereto is confirmatory of the presence of, or infection with, Mycobacterium bovis.

Where the selected polypeptide is present in the wild-type Mycobacterium but not in strains used for vaccines, such as Myobacterium bovis bacillus Calmette-Guerin (BCG), the invention may be used to discriminate between infected animals and vaccinated animals. This will be particularly valuable in the control of tuberculosis in herds of animals.

Accordingly, in a third aspect of the invention, there is provided a method of determining whether an animal is infected with tuberculosis or vaccinated against tuberculosis, said method comprising the steps:

(a) providing a biological sample from said animal; and
(b) determining the presence or absence in the biological sample of:
(i) at least three of the group consisting of an Rv3616 antigen, an MPB70 antigen, and MPB83 antigen, and an α crystalline 2 antigen, in the biological sample or an immune response thereto; and
(ii) two or more of the group consisting of an ESAT6 antigen, a CFP10 antigen, a PE35 antigen, and a PPE68 antigen or an immune response thereto;
wherein the identification of the presence of (i) at least one of the group consisting of an Rv3616 antigen, an MPB70 antigen, and MPB83 antigen, and an α crystalline 2 antigen, or an immune response thereto, and (ii) at least one of the group consisting of an ESAT6 antigen, a CFP10 antigen, a PE35 antigen, and a PPE68 antigen, or an immune response thereto is indicative of the presence of infection of the animal with tuberculosis; and
wherein the identification of the presence of at least one of the group consisting of an Rv3616 antigen, an MPB70 antigen, and MPB83 antigen, and an a crystalline 2 antigen, or an immune response thereto, combined with the absence of each of the antigens tested of (ii), or an immune response thereto, is indicative of vaccination of the animal with a vaccine.

Of particular utility in this aspect of the invention is the identification of the presence of one or more ESAT6 antigen, a CFP10 antigen, a PE35 antigen, and a PPE68 antigen, or of an immune response to said antigens. These antigens are not present in BOG vaccines and thus detection of their presence, or of an immune response thereto, is indicative of a source of antigen, such as infection, other than such vaccination. Thus the presence of one or more of said antigens, or the presence of an immune response thereto, is indicative that the animal is infected with tuberculosis.

In the methods of the invention, the inventors have determined that, by testing for presence of (i) seven or more of the group of antigens consisting of an Rv3616 antigen, an MPB70 antigen, and MPB83 antigen, an ESAT 6 antigen, a CFP10 antigen, an α crystalline 0.2 antigen, a PE35 antigen, and a PPE68 antigen, or (ii) the group of antigens consisting of an Rv3616 antigen, an MPB70 antigen, an MPB83 antigen, an ESAT 6 antigen, the sensitivity of testing for the presence of Mycobacteria and of tuberculosis infection is greatly improved.

The methods of the invention may be used with any animal, for example any mammal, including humans or bird. In one embodiment, the animal is a mammal. In one particular embodiment, the mammal is a non-human mammal.

In particular embodiment of the invention, the mammal is a cow.

A number of wild mammals are considered reservoirs for TB infection. For example, badgers are considered as a significant source of TB infection for cattle and, as a result, are often culled to prevent TB spread to cattle. However, this means of control of TB infection is controversial. A test which could reliably identify TB infection in badgers (and other host animals, which may act as reservoirs of infection) would therefore be invaluable in establishing whether or not individual animals, sets or populations thereof are indeed a threat to other species and thus save unnecessary culling. To date, however, a test which can reliably identify infection in animals such as badgers is not available. Typical tests have a reported sensitivity of less than 50% when assessing field samples (Chambers et al J Clin Microbiol. 2008 April; 46(4):1498-500).

The methods of the present invention achieve considerably higher sensitivity than known tests for use in diagnosis of TB infection in animals such as badgers. Thus, in one embodiment of the invention, the mammal is a “reservoir” animal, for example a badger. In the context of the present invention, a “reservoir” animal is any non-human animal which may carry or be infected with tuberculosis and which may act as a source of infection of tuberculosis to domesticated livestock. Such reservoir animals include, in addition to badgers, white tail deer, possums, elephant and zoo/safari park animals.

Moreover, the inventors have determined that a considerable improvement in sensitivity may be achieved over known reservoir animals, e.g. badger, TB diagnostic assays by testing for the presence or absence of, or an immune response to, MPB70, Rv3616c and MPB83.

Accordingly, in a fourth aspect of the invention, there is provided a method of diagnosis of tuberculosis in a mammal, the method comprising the steps:

providing a biological sample from said mammal; and determining the presence or absence of, or an immune response to an Rv3616 antigen, an MPB70 antigen, and an MPB83 antigen, wherein the identification of the presence of one or more of said antigens of said group of antigens, or of an immune response thereto, is indicative of the presence of tuberculosis infection in the mammal.

In one embodiment of the fourth aspect of the invention, the mammal is a “reservoir” mammal. In a particular embodiment, the mammal is a badger.

In the present application, unless the context demands otherwise, references to tuberculosis should be taken to refer to any tuberculosis, for example human tuberculosis, non-human animal tuberculosis, avian tuberculosis or a para-tuberculosis disease, such as Johne's disease.

Unless the context demands otherwise, reference to Mycobacterium should be taken to refer to any Mycobacterium.

In one embodiment, the Mycobacterium is Mycobacterium tuberculosis.

In another embodiment the Mycobacterium is Mycobacterium bovis.

In another embodiment, the Mycobacterium is Mycobacterium avium.

In another embodiment, the Mycobacterium is Mycobacterium paratuberculosis.

Any suitable biological sample may be used in the methods of the invention. Suitable biological samples include but are not limited to whole blood, serum, plasma, saliva, cerebrospinal fluid, urine and tissue samples.

In the methods of the invention, the presence of particular antigens or an immune response thereto may be determined using any means known in the art, either directly or indirectly. For example, in one embodiment, the presence of the antigen in the sample is determined; alternatively or additionally the presence of an antibody specific to said antigen is determined; alternatively, or additionally, the presence of an nucleic acid encoding said antibody or said antigen is determined.

The identification by the inventors of the particular selection of mycobacterial antigens which are particularly immunogenic, which are widely prevalent across geographical areas, and which moreover, cover each of the different stages of the TB life cycle enables the use of these polypeptides in the preparation of novel vaccines against tuberculosis. Such vaccines should provide a vaccinated animal with a sustained resistance to infection.

Accordingly, in a fifth aspect of the present invention, there is provided a vaccine or immunomodulator comprising at least seven of the antigens, or variants or fragments thereof, of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

In one embodiment of the fifth aspect of the invention the vaccine comprises an antigen (or variant or fragment thereof) of each member of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

Given the demonstration of the high sensitivity of detection of TB infection in reservoir animals such as badgers when testing for the presence or absence of, or an immune response to, MPB70, Rv3616c and MPB83, it is believed that vaccines comprising at least these three antigens would be expected to have a significant effect on protecting against TB infection in such reservoir animals.

Thus, in a sixth aspect of the present invention, there is provided a vaccine or immunomodulator for reservoir animals, said vaccine or immunomodulator comprising an Rv3616 antigen or variant or fragment thereof, an MPB70 antigen or variant or fragment thereof, and an MPB83 antigen or variant or fragment thereof.

In one embodiment of the fifth or sixth aspects of the invention, the vaccine is a vaccine against tuberculosis.

In an alternative embodiment of the fifth or sixth aspects of the invention, one or more of said polypeptides are used in a vaccine as an adjuvant.

In a seventh aspect of the invention, there is provided a method of providing immunity in an animal against tuberculosis, the method comprising administering to said animal a vaccine according to the fifth aspect or the sixth aspect of the invention.

In an eighth aspect of the invention, there is provided the use of at least seven of the antigens, or variants or fragments thereof, of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen in the preparation of a vaccine against tuberculosis.

In a ninth aspect, the invention provides a diagnostic kit for the diagnosis of the presence of tuberculosis in a subject, said kit comprising:

at least seven, such as eight, of the antigens (or variants or fragments thereof) of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

In one embodiment of the ninth aspect of the invention, the kit comprises each of the eight antigens (or variants or fragments thereof).

In a tenth aspect of the invention, there is provided a diagnostic kit for the diagnosis of the presence of tuberculosis in a subject, said kit comprising an Rv3616 antigen, an MPB70 antigen, an MPB83 antigen, and an ESAT 6 antigen.

In one embodiment, the MPB70 antigen is a full length MPB70 protein. In another embodiment, the MPB70 antigen is an MPB70 linear epitope, such as the PEP1 (MPB70) antigen. In a further embodiment of this aspect of the invention, the kit comprises both a full length MPB70 protein and an MPB70 linear epitope.

In an embodiment of the ninth or tenth aspect of the invention, the kit further comprises at least one of the antigens selected from the group consisting of an Rv1573 antigen, an Rv1580c antigen or an Rv1585c antigen.

In an eleventh aspect of the invention, there is provided a diagnostic kit for the diagnosis of the presence of tuberculosis in a biological sample, said kit comprising:

at least seven of the group consisting of an antibody molecule with binding specificity for an Rv3616 antigen, an antibody molecule with binding specificity for an MPB70 antigen, an antibody molecule with binding specificity for an MPB83 antigen, an antibody molecule with binding specificity for an ESAT 6 antigen, an antibody molecule with binding specificity for a CFP10 antigen, an antibody molecule with binding specificity for an α crystalline 2 antigen, an antibody molecule with binding specificity for a PE35 antigen, and an antibody molecule with binding specificity for a PPE68 antigen.

In one embodiment of the eleventh aspect of the invention, the kit comprises eight of the recited group of antibody molecules.

In an twelfth aspect of the invention, there is provided a diagnostic kit for the diagnosis of the presence of tuberculosis in a biological sample, said kit comprising: an antibody molecule with binding specificity for an Rv3616 antigen, an antibody molecule with binding specificity for an MPB70 antigen, an antibody molecule with binding specificity for an MPB83 antigen.

In one embodiment, the MPB70 antigen is a full length MPB70 protein. In another embodiment, the MPB70 antigen is an MPB70 linear epitope, such as the PEP1 (MPB70) antigen. In a further embodiment of this aspect of the invention, the kit comprises both an antibody molecule with binding specificity for full length MPB70 protein and an antibody molecule with binding specificity for an MPB70 linear epitope.

In an embodiment of the eleventh or twelfth aspect, the kit further comprises at least one of the antibody molecules selected from the group consisting of an antibody molecule with binding specificity for an Rv1573 antigen, an antibody molecule with binding specificity for an Rv1580c antigen, and an antibody molecule with binding specificity for an Rv1585c antigen.

In a thirteenth aspect of the invention, there is provided a diagnostic kit for the diagnosis of the presence of tuberculosis in a reservoir animal such as a badger, said kit comprising an Rv3616 antigen, an MPB70 antigen, and an MPB83 antigen.

In a fourteenth aspect of the invention, there is provided a diagnostic kit for the diagnosis of the presence of tuberculosis in a biological sample from a reservoir animal such as a badger, said kit comprising: an antibody molecule with binding specificity for an Rv3616 antigen, an antibody molecule with binding specificity for an MPB70 antigen, and an antibody molecule with binding specificity for an MPB83 antigen.

Preferred features of each aspect of the invention are as for each of the other aspects mutatis mutandis.

### DETAILED DESCRIPTION

### Polypeptides, Variants and Fragments

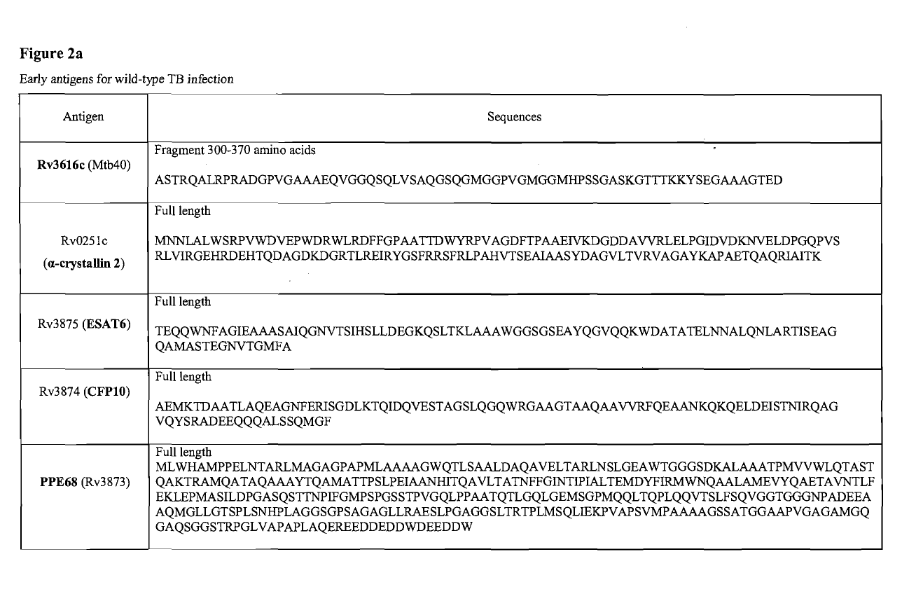

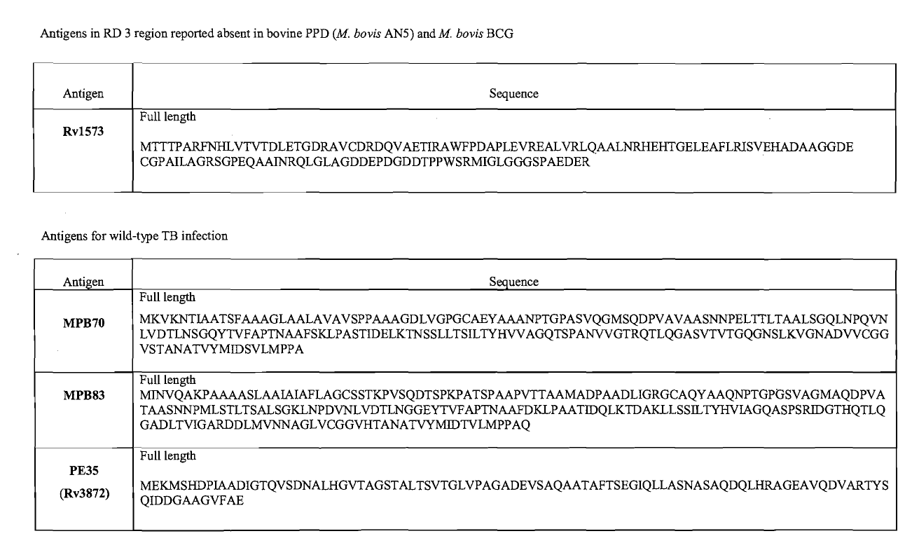

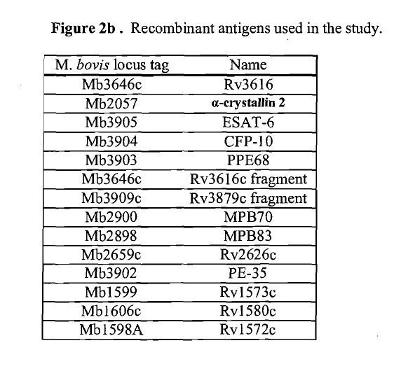

As described above, the inventors have identified a number of tuberculosis antigens which may be used in the diagnosis of tuberculosis, the differentiation between strains of Mycobacteria, and/or preparation of vaccines. The antigens comprise an Rv3616 antigen, an MPB70 antigen, and MPB83 antigen, an ESAT 6 antigen, a CFP10 antigen, an α crystalline 2 antigen, a PE35 antigen, and a PPE68 antigen. Polypeptides sequences for each of these antigens are provided in FIG. 2. In one embodiment of the invention the full length antigen sequences are used. However, fragments may also be used. For example, with respect to the rv3616c, examples of particular fragments which may be used are provided as SEQ ID Nos 2-5. In one embodiment of the invention, the antigens for use in the invention consist of polypeptides consisting of the amino acid sequence shown as any one of SEQ ID NO:1 to SEQ ID NO: 26.

However, the present invention is not limited to the use of polypeptides having such specific sequences of the polypeptides or antibodies disclosed herein but also extends to variants thereof. Thus, variant polypeptide sequences in which one or more amino acid residues are modified may also be used as the polypeptides in the invention. For example such variants may be useful in the preparation of vaccines or the raising of antibodies which may be used in kits of the invention. Modifications may involve insertion, addition, deletion and/or substitution of one or more amino acids. The modified amino acid residues in the amino acid sequences of the variant are preferably 30% or less, more preferably 20% or less, most preferably 10% or less, within the entire polypeptide. Such variants may be provided using the teaching of the present application and techniques known in the art.

Preferably such variants involve the insertion, addition, deletion and/or substitution of 15 or fewer amino acids, more preferably of 10 or fewer, even more preferably of 5 or fewer, most preferably of 1 or 2 amino acids only.

Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions may include but are not limited to Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu.

In preferred embodiments, a variant or fragment retains the immune reactivity of the polypeptide having the amino acid sequence shown as any one of SEQ ID NO:1 to SEQ ID NO: 26, of which it is a variant or fragment.

For the avoidance of any doubt, in the present application, where reference is made to the presence of two or more polypeptides selected from a list, for example two or more of the polypeptides selected from the polypeptides having the amino acid sequence shown as any one of SEQ ID NO:1, SEQ ID NO:2, SEQ ID NO:3, etc, it should be understood that reference is being made to at least one (first) polypeptide having one of the recited amino acid sequences and at least one (second) polypeptide having another of the recited amino acid sequences, which is different from the amino acid sequence of the first polypeptide.

Furthermore, for the avoidance of any doubt, in the present application, unless the context demands otherwise, where reference is made to the determination of the presence or absence of two or more polypeptides/antigens selected from a list, or the presence or absence of an immune response thereto, it should be understood that such a statement encompasses the determination of the presence or absence of two or more of said polypeptides/antigens selected from the list or the determination of the presence or absence of an immune response to two or more of said polypeptides/antigens or indeed a mixture thereof i.e. the determination of the presence or absence of one or more polypeptides/antigens selected from the list combined with the determination of an immune response to one or more other polypeptides/antigens selected from the list.

Furthermore for the avoidance of any doubt, where reference is made to MPB70 or variants thereof and pep1 (MPB70) together, pep1 (MPB70) should not be considered as an MPB70 variant.

### Antibody Molecules

In the context of the present invention, an “antibody molecule” should be understood to refer to an immunoglobulin or part thereof or any polypeptide comprising a binding domain which is, or is homologous to, an antibody binding domain. Antibodies include but are not limited to polyclonal, monoclonal, monospecific, polyspecific antibodies and fragments thereof and chimeric antibodies comprising an immunoglobulin binding domain fused to another polypeptide.

Intact (whole) antibodies comprise an immunoglobulin molecule consisting of heavy chains and light chains, each of which carries a variable region designated VH and VL, respectively. The variable region consists of three complementarity determining regions (CDRs, also known as hypervariable regions) and four framework regions (FR) or scaffolds. The CDR forms a complementary steric structure with the antigen molecule and determines the specificity of the antibody.

Fragments of antibodies may retain the binding ability of the intact antibody and may be used in place of the intact antibody. Accordingly, for the purposes of the present invention, unless the context demands otherwise, the term “antibodies” should be understood to encompass antibody fragments. Examples of antibody fragments include Fab, Fab′, F(ab′)₂, Fd, dAb, and Fv fragments, scFvs, bispecific scFvs, diabodies, linear antibodies (see U.S. Pat. No. 5,641,870, Example 2; Zapata et al., Protein Eng 8 (10): 1057-1062 [1995]); single-chain antibody molecules; and multispecific antibodies formed from antibody fragments.

The Fab fragment consists of an entire L chain (VL and CL), together with VH and CH1. Fab′ fragments differ from Fab fragments by having an additional few residues at the carboxy terminus of the CH1 domain including one or more cysteines from the antibody hinge region. The F(ab′)2 fragment comprises two disulfide linked Fab fragments.

Fd fragments consist of the VH and CH1 domains.

Fv fragments consist of the VL and VH domains of a single antibody.

Single-chain Fv (scFv) fragments are antibody fragments that comprise the VH and VL domains connected by a linker which enables the scFv to form an antigen binding site (see Pluckthun in The Pharmacology of Monoclonal Antibodies, vol. 113, Rosenburg and Moore eds., Springer-Verlag, New York, pp. 269-315 (1994).

Diabodies are small antibody fragments prepared by constructing scFv fragments with short linkers (about 5-10 residues) between the VH and VL domains such that inter-chain but not intra-chain pairing of the V domains is achieved, resulting in a multivalent fragment, i.e. a fragment having two antigen-binding sites (see, for example, EP 404 097; WO 93/11161; and Hollinger et al., PNAS. USA, 90: 6444-6448 (1993)).

Further encompassed by fragments are individual CDRs. The CDRs may be carried on a framework structure comprising an antibody heavy or light chain sequence or part thereof. Preferably such CDRs are positioned in a location corresponding to the position of the CDR(s) of naturally occurring VH and VL domains. The positions of such CDRs may be determined as described in Kabat et al, Sequences of Proteins of Immunological Interest, US Dept of Health and Human Services, Public Health Service, Nat'l Inst. of Health, NIH Publication No. 91-3242, 1991 and online at www.kabatdatabase.com http://immuno.bme.nwu.edu. Furthermore, modifications may alternatively or additionally be made to the Framework Regions of the variable regions. Such changes in the framework regions may improve stability and reduce immunogenicity of the antibody.

The antibody molecules for use in the present invention extend, for example, to any other antibody which specifically binds a polypeptide identified herein as inducing a strong immune response i.e. an antibody molecule which retains binding specificity for a polypeptide consisting of amino acid sequence shown as any one of SEQ ID NOs:1 to 26.

Antibodies for use in the invention herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., PNAS. USA, 81: 6851-6855 (1984)). Chimeric antibodies of interest herein include “primatized” antibodies comprising variable domain antigen-binding sequences derived from a non-human primate (e.g. Old World Monkey, Ape etc), and human constant region sequences.

Antibody molecules for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352: 624-628 and Marks et al. (1992) Bio/Technology, 10: 779-783). Other antibody production techniques are described in Antibodies: A Laboratory Manual, eds. Harlow et al., Cold Spring Harbor Laboratory, 1988.

Traditional hybridoma techniques typically involve the immunisation of a mouse or other animal with an antigen in order to elicit production of lymphocytes capable of binding the antigen. The lymphocytes are isolated and fused with a myeloma cell line to form hybridoma cells which are then cultured in conditions which inhibit the growth of the parental myeloma cells but allow growth of the antibody producing cells. The hybridoma may be amenable to genetic mutation, which may or may not alter the binding specificity of antibodies produced. Synthetic antibodies can be made using techniques known in the art (see, for example, Knappik et al, J. Mol. Biol. (2000) 296, 57-86 and Krebs et al, J. Immunol. Meth. (2001) 2154 67-84).

Modifications may be made in the VH, VL or CDRs of the antibody molecules, or indeed in the FRs using any suitable technique known in the art. For example, variable VH and/or VL domains may be produced by introducing a CDR, e.g. CDR3 into a VH or VL domain lacking such a CDR. Marks et al. (1992) Bio/Technology, 10: 779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences of the present invention may be produced.

Alternative techniques of producing antibodies for use in the invention may involve random mutagenesis of gene(s) encoding the VH or VL domain using, for example, error prone PCR (see Gram et al, 1992, PNAS 89 3576-3580. Additionally or alternatively, CDRs may be targeted for mutagenesis e.g. using the molecular evolution approaches described by Barbas et al 1991 PNAS 3809-3813 and Scier 1996 J Mol Biol 263 551-567.

Having produced such variants, antibodies and fragments they may be tested for binding to Mycobacterium bovis.

The antibodies for use in the invention may comprise further modifications. For example the antibodies can be glycosylated, pegylated, or linked to albumin or a nonproteinaceous polymer.

Antibodies for use in the invention may be labelled. Labels which may be used include radiolabels, enzyme labels such as horseradish peroxidase or alkaline phosphatase, or biotin.

### Nucleic Acid

Nucleic acid for use in the present invention may comprise DNA or RNA. It may be produced recombinantly, synthetically, or by any means available to those in the art, including cloning using standard techniques.

The nucleic acid may be inserted into any appropriate vector. A vector comprising a nucleic acid of the invention forms a further aspect of the present invention. In one embodiment the vector is an expression vector and the nucleic acid is operably linked to a control sequence which is capable of providing expression of the nucelic acid in a host cell. A variety of vectors may be used. For example, suitable vectors may include viruses (e.g. vaccinia virus, adenovirus, etc.), baculovirus); yeast vectors, phage, chromosomes, artificial chromosomes, plasmids, or cosmid DNA.

The vectors may be used to introduce the nucleic acids of the invention into a host cell. A wide variety of host cells may be used for expression of the nucleic acid of the invention. Suitable host cells for use in the invention may be prokaryotic or eukaryotic. They include bacteria, e.g. E. coli, yeast, insect cells and mammalian cells. Mammalian cell lines which may be used include Chinese hamster ovary cells, baby hamster kidney cells, NSO mouse melanoma cells, monkey and human cell lines and derivatives thereof and many others.

A host cell strain that modulates the expression of, modifies, and/or specifically processes the gene product may be used. Such processing may involve glycosylation, ubiquiination, disulfide bond formation and general post-translational modification.

For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 5th ed., Ausubel et al. eds., John Wiley & Sons, 2005 and, Molecular Cloning: a Laboratory Manual: 3^{rd} edition Sambrook et al., Cold Spring Harbor Laboratory Press, 2001.

### Diagnostic Methods, Assays and Kits

The invention may be used in the diagnosis of a variety of conditions and disorders associated with tuberculosis. These include Mycobacterium bovis, Mycobacterium avium and human mycobacterium and para-tuberculosis diseases such as Johne's disease.

In using the methods of the invention to identify the infection with a mycobacterial strain, either as current or previous infection, the presence or absence of immunogenic polypeptides, or the presence or absence of an immune response to said polypeptides is determined from a biological sample. Any suitable biological sample may be used. For example, the biological sample may be a biological fluid, such as sputum, saliva, plasma, blood, urine or sperm, or a tissue, such as a biopsy of a tissue.

Diagnostic and assay means of detecting the presence of polypeptides or an immune responses to said polypeptides are known in the art. For example, the presence of the polypeptides may be detected by use of antibodies specific to said polypeptides.

Alternatively, using standard techniques in the art, the presence of nucleic acids encoding the polypeptide or indeed an antibody specific to said polypeptide may be used. Further, the presence of antibodies specific to said polypeptides may be used to determine the presence of an immune response to said polypeptide.

Techniques which may be employed include but are not limited to ELISA, Immunohistochemistry, Electron Microscopy, Latex agglutination, Immuno Blotting, immunochromatography, immunochips, lateral flow immunoassays and Dip Stick Immuno testing.

The ELISA test (enzyme linked immunoenzymatic assay) is frequently used for serological diagnosis. This method allows the identification and quantification of antigens or antibodies in biological fluids. The conventional ELISA consists in the detection of the complex antibody-antigen by a second antibody (against the antibody that reacts with the antigen) conjugated to an enzymatic activity (peroxidase, alkaline phosphatase and others).

In the latex agglutination assay, the antigen preparation is affixed to latex beads. The biological sample is then incubated directly on a slide with the latex particles. In a short time the reaction is examined for the presence of cross-linked, or agglutinated latex particles indicating the presence of antibodies to polypeptides in the sample.

Immunochips may used to determine the presence of the specific Mycobacterium antigens. Generally, the specific antibodies to the antigens are immobilised on a transducer, e.g. electrodes, caloric meter, piezoelectric crystal, surface plasmon resonance transducer, surface acoustic resonance transducer or other light detecting device. The binding of antigens in the biological sample to the immobilised specific antibody is detected by a change in electric signal.

As described above, the presence of the immunogenic antigens may be detected by detecting nucleic acids encoding the antigen or encoding antibodies raised against the antigen. Such techniques are well known in the art. For example, where large amounts of DNA are available, genomic DNA may be used directly. Alternatively, the region of interest is cloned into a suitable vector and grown in sufficient quantity for analysis. The nucleic acid may be amplified by conventional techniques, such as the polymerase chain reaction (PCR) (Saiki, et al. (1985) Science 239:487). Primers may be used to amplify sequences encoding the polypeptide of interest. Optionally, a detectable label, for example a fluorochrome, biotin or a radioactive label may be used in such an amplification reaction. The label may be conjugated to one or both of the primers. Alternatively, the pool of nucleotides used in the amplification is labeled, so as to incorporate the label into the amplification product.

The sample nucleic acid, e.g. amplified or cloned may be analysed using any suitable method known in the art. For example, the nucleic acid may be sequenced by dideoxy or other methods, and the sequence of bases compared to the deleted sequence. Hybridization with the variant sequence may also be used to determine its presence, by Southern blots, dot blots, etc. The hybridization pattern of a control and variant sequence to an array of oligonucleotide probes immobilized on a solid support, as described in WO95/35505, may be used as a means of detecting the presence or absence of a sequence.

In one embodiment, the kit contains an antigen preparation prepared as described above and then fixed onto a solid support for use in a serological assay. The kit may also contain an explanatory note on how to proceed.

In one embodiment, the assay is in a dip stick or test strip format. Such assays typically employ a solid support medium, which is able to bind target substances such as proteins, for example protein antigens. In use, the test strip is exposed to a sample, for example serum, such that antibodies in the serum may bind target substances such as antigens on the test strip. After washing, the dip stick/test strip is exposed to a labelled probe which binds the bound antibody of interest, with binding typically visualised by means of further incubation with a visualisable substrate, for example a chromogenic substrate.

In a further embodiment, the assay of the invention is provided as a lateral flow immunoassay. Such devices are well known in the art. Lateral flow immunoassay devices typically employ a microporous element along which a sample may flow laterally, the device having a capture region for binding an analyte of interest in the sample. Generally, the lateral flow device has an application zone, to which a sample is applied, a conjugate zone, containing a mobile reporter conjugate, which is typically a visually observable reporter e.g. colloidal gold conjugated to an antibody directed against the analyte and a capture region comprising a capture agent which binds the analyte. In use, the sample is applied to the application zone and flows along a strip to a first region, at which the reporter conjugate binds the analyte. Thereafter, movement of the reporter conjugate/analyte complex to the capture region results in capture of the complex at that region. Uncomplexed reporter conjugate does not bind at the capture region. By determining the amount of visually detectable signal at the capture region, the presence of analyte in a sample may be determined. Variants of such kits are well known in the art.

In one embodiment of the invention, the methods, assays and kits of the invention comprise or use an assay in which the presence or absence of a number of antigens or n immune response thereto is analysed simultaneously, i.e. using a multiplex based assay. Any suitable multiplex assay may be used. For example, arrays of different tuberculosis antigens may be provided in microplate wells and a sample mixed with reporter antibodies in the wells. Other multiplex based methods may involve protein arrays placed on a matrix, with the response to individual proteins on the solid-phase array assayed. The use of such multiplex techniques enables the rapid identification of the presence or absence of a plurality of target antigens (or antibodies) during a single run of the analysis.

As described in the Examples, the inventors have developed a multiplex technique in which a plurality of different antigens, for example up to 25, are spotted in a single well of a 96 well plate. Surprisingly, the inventors have shown that high sensitivity of reaction with each of the antigens tested was possible in the multiplex reactant mixture despite the necessary requirement of a single buffer being used for each of the plurality of antigen—antibody reactions in a single well.

Using such a plate, up to 96 samples, each from a different animal, may be tested on a single plate with the each samples reactivity with each of the antigens measured simultaneously. Such a technique therefore enables the rapid analysis of infection with, for example, tuberculosis, in a large number of animals simultaneously.

Moreover, the inventors have surprisingly found that the advantages of such a system for the determination of tuberculosis infection is not just with respect to the convenience of being able to test many different antigens in many different samples simultaneously. To their surprise, the inventors have also demonstrated that the multiplex platform provided higher sensitivity and specificity than that obtained using conventional assays such as lateral flow assays and ELISA.

Accordingly, in one aspect of the invention, there is provided a multiplex method of diagnosis of tuberculosis in an animal, the method comprising the steps:

providing a substrate on which is immobilised a plurality of different antigens, bringing into contact with said substrate a biological sample from an animal,
identifying the presence or absence of binding of antibodies from said sample with said antigens,
wherein the determination of binding is indicative of the presence of tuberculosis infection in the animal.

In one embodiment, the substrate is a microplate. In one such embodiment, a plurality of different antigens are immobilised in a single well of a plate. In one embodiment of this aspect of the invention, the plurality of antigens comprise two or more, for example more than 5, 10, 15 or 20 of the antigens having amino acid sequences SEQ ID NO: 1 to SEQ ID NO: 26, or fragments or variants thereof.

In a further aspect of the invention, there is provided a multiplex method of diagnosis of tuberculosis in an animal, the method comprising the steps:

providing a substrate to which is attached a plurality of different antibodies,
bringing into contact with said substrate a biological sample from an animal,
identifying the presence or absence of binding of antigens from said sample with said antibodies,
wherein the determination of binding is indicative of the presence of tuberculosis infection in the animal.

In one embodiment, the substrate is a microplate. In one such embodiment, a plurality of different antibodies are immobilised in a single well of a plate. In one embodiment of this aspect of the invention, the plurality of antibodies comprises two or more, for example more than 5, 10, 15 or 20 antibodies each having binding specificity for a different member of the group of antigens having amino acid sequences Sequence ID No: 1 to Sequence ID No: 26.

The kit can be used to perform the methods of the invention described above.

### Vaccines

As described above, the present invention also extends to vaccines for use in protecting against tuberculosis and tuberculosis-associated diseases.

One way of making a vaccine according of or for use in the invention is by biochemical purification of the immunogenic polypeptides from bacteria.

Alternatively, expression products of the genes encoding the polypeptides according to the invention may be used in vaccines. Such vaccines based upon the expression products of these genes can easily be made by admixing one or more proteins with a pharmaceutically acceptable carrier.

Alternatively, a vaccine according to the invention can comprise live recombinant carriers, capable of expressing the polypeptides according to the invention.

Vaccines described above all contribute to active vaccination, i.e. the host's immune system is triggered by one or more proteins according to the invention or immunogenic fragments thereof, to make antibodies against these proteins.

Alternatively, such antibodies can be raised in e.g. rabbits or can be obtained from antibody-producing cell lines. Such antibodies can then be administered to the host animal. This method of vaccination, passive vaccination, is the vaccination of choice when an animal is already infected, and there is no time to allow the natural immune response to be triggered. It is also the preferred method for vaccinating immune-compromised animals.

Therefore, one other form of this embodiment of the invention relates to vaccines comprising antibodies against one or more of the immunogenic polypeptides used in the invention.

An alternative and efficient way of vaccination is direct vaccination with DNA encoding the relevant antigen. Direct vaccination with DNA encoding proteins has been successful for many different proteins. (As reviewed in e.g. Donnelly et al., The Immunologist 2:20-26 (1993)). Therefore, still other forms of this embodiment of the invention relate to vaccines comprising nucleic acid sequences encoding a polypeptide as used in the invention or immunogenic fragments thereof, and to vaccines comprising DNA fragments that comprise such nucleic acid sequences.

Still other forms of this embodiment relate to vaccines comprising recombinant DNA molecules according to the invention.

DNA vaccines can easily be administered through intradermal application e.g. using a needle-less injector.

Vaccines according to the present invention may comprise a pharmaceutically acceptable carrier e.g. sterile water, a sterile physiological salt solution, or a buffer, and may also contain an adjuvant.

The invention will now be described further in the following non-limiting examples. Reference is made to the accompanying drawings in which:

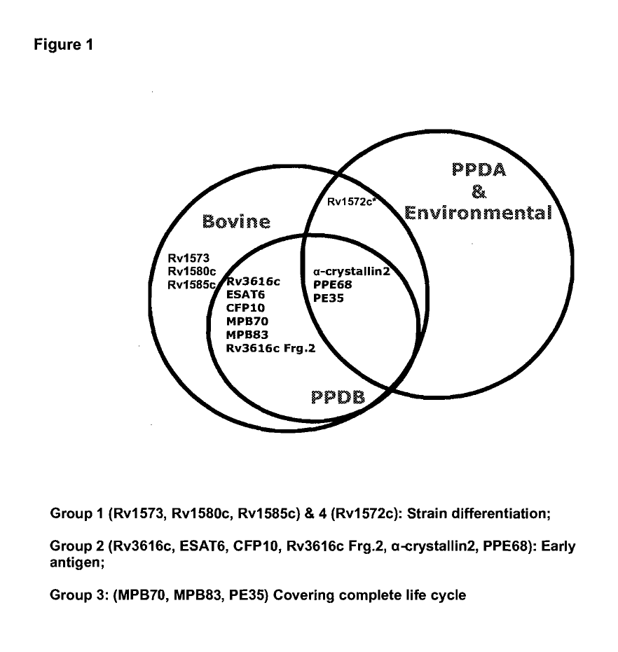

FIG. 1 shows a Venn diagram illustrating the expression of antigens between different Mycobacterium strains; Group 1 (strain differentiation)—Rv1573, RV1580c, Rv1585c, Rv1572c; Group 2 (Early antigen)—Rv3616c, ESAT6, CFP10, alpha-crystallin 2, PPE68; Group 3 (covering complete life cycle)—MPB70, MPB83, and PE35.

FIG. 2(a) shows the amino acid sequences of antigens used in the present invention.

FIG. 2(b) shows recombinant antigens used in the multiplex assay.

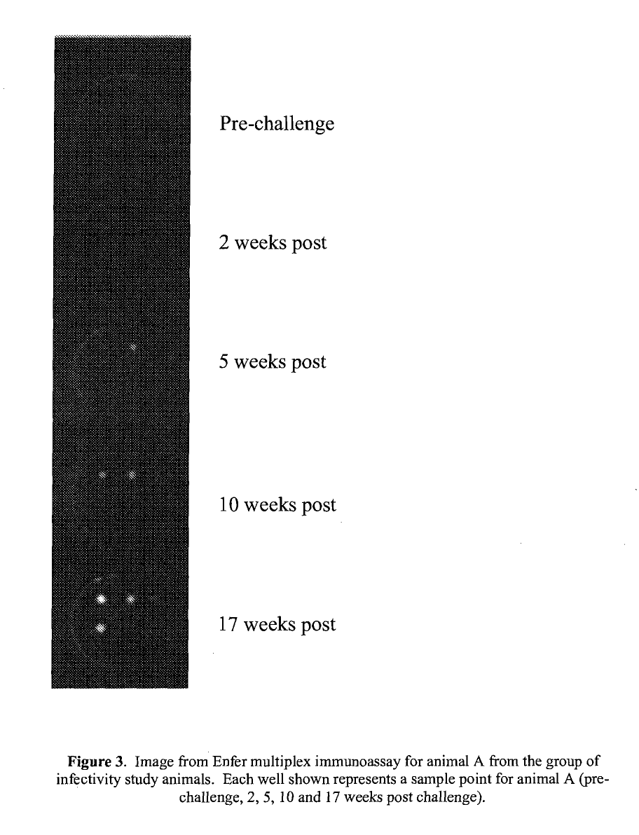

FIG. 3 FIG. 3 shows an example of the captured image of the test wells from the multiplex ELISA for one of the animals from the infectivity study. Each well shown represents a sample point for animal A (pre-challenge, 2, 5, 10 and 17 weeks post challenge).

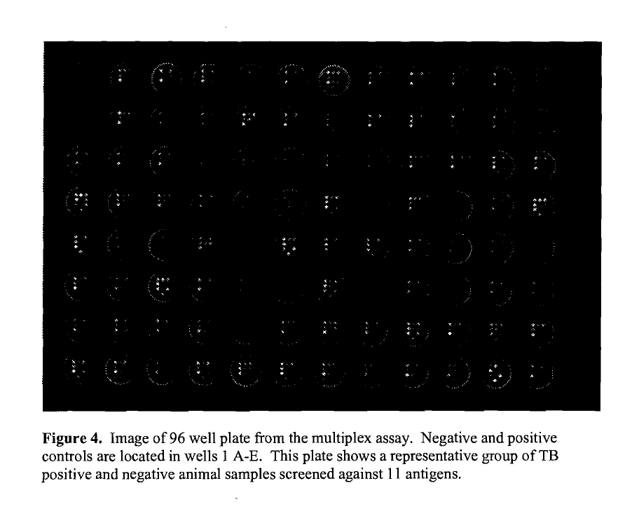

FIG. 4 illustrates an image of 96 well plate from the multiplex assay. Negative and positive controls are located in wells 1 A-E. This plate shows a representative group of TB positive and negative animal samples screened against 11 antigens.

### EXAMPLE 1

The inventors have used bioinformatics software to identify and characterise proteins which are specific to one or other mycobacterial strains. Using a genome alignment strategy the group has identified point mutations or deletions which allow differentiation between M bovis, PPD (M. bovis AN5 and M. avium), M. bovis BCG and environmental mycobacteria (FIG. 1). They have identified a panel of proteins capable of strain identification (FIG. 2(a).

As described below, the inventors have surprisingly found that by using a panel of seven or eight of these antigens, compared to known techniques, the accuracy of detection of tuberculosis may be greatly improved.

Significantly, a high degree of sensitivity demonstrated using this particular panel of antigens is obtainable across cohorts of tested animals from different geographical regions, giving a level of coverage which is unprecedented in tuberculosis testing.

### Methodology

### Cloning, Expression and Purification of Mycobacterial Antigens

DNA encoding target mycobacterial antigens (FIG. 2(a)) were identified and analysed by bioinformatics software. Primers were designed to amplify selected antigenic fragments of each of the target antigen by PCR. The PCR mixture contained 10 ng M. tuberculosis genomic DNA as template in a total volume of 50 μl with 47 μl Tag polymerase supermix (1 Unit of recombinant enzyme), and 1 μl of each primer (10 pMol). The amplification was carried out with initial heating at 95° C. for 4 min followed by 25 cycles of denaturation at 95° C. for 30 s, annealing at 55° C. for 30 s, and extension at 72° C. for 1 min in each cycle. The final polymerisation was carried out for 10 min. The PCR products were analysed by 1.5% agarose gel electrophoresis, and purified. Each PCR product after purification was digested with appropriate enzymes and cloned in respective FET expression vectors with C-terminal (His6) tag. The resultant constructs were characterised by sequencing using Big dye terminator chemistry and automated DNA sequencer (ABI Prism 3100).

Recombinant mycobacterial antigens were expressed by FET system. Competent e-coli cells were transformed with various recombinant plasmids. The successful transformed cells were picked and grown into 5 ml LB containing 100 μg/ml of ampicillin at 37° C. with shaking overnight. 100 μl of this culture was used to inoculate a 500 ml of pre-warmed medium with antibiotics listed above and cultured at 37° C. for 120 min at 300 rpm. Expression of recombinant antigen was induced from the cells at this time with 1 μl/ml of 25 mM IPTG, and the cells cultured for a further 4 hours. The cells were then pelleted by centrifugation for 15 min and the pellets were resuspended in 100 ml 8 M urea and lysed overnight at room temperature with shaking. The lysates were analysed by SDS-PAGE for presence of recombinant mycobacterial antigens.

Recombinant mycobacterial antigens were purified by using FET purification system. Antigen fragments were purified from the lysates using affinity chromatography. The elution was monitored at 280 nm and 2 ml fractions were collected. The fractions were analysed by SDS-PAGE followed by Commassie/Sliver staining and Western Blot.

### ELISA Assays

Part 1: Plate Coating
1. The plate frames were labelled with protein name and date of coating
2. The plates were coated, at the relevant protein concentration (the protein is diluted in the coating buffer), by adding 100 μl/well.
3. The plates were covered with a plate sealer and incubated at 2-8° C. on a level surface overnight.
4. The plates were washed with EW2 (1×) and tapped dry on tissue paper

Part 2: Plate Blocking
5. The plates were blocked by adding 200 μl of the Blocking Buffer to each well.
6. The plates were covered with a plate sealer.
7. The plates were incubated for 30 minutes at 37° C. Stationary.
8. The plates were washed with EW2 and tapped dry on tissue paper.

Part 3: The Assay
9. The sample was diluted to a 1:250 dilution in the Sample Dilution Buffer). Sample dilutions were carried out on the Tecan Genesis automated system or manually.
10. Diluted samples were transferred to the test plates on the Tecan Genesis or manually −100 μl of sample is added to each well of the test plate according to the plate layout required.
11. The plate was covered with a plate sealer and incubated at 34° C. Shake Speed 3 for 30 min.
12. The plate was washed with EW2 and tapped dry on tissue paper.
13. The conjugate was prepared to a 1:2500 dilution in the conjugate dilution buffer
14. 100 μl of conjugate was added to each well on the Tecan Genesis or manually.
15. The plate was covered with a plate sealer and incubated at 34° C. Shake Speed 3 for 1 hour.
16. The plate was washed with EW2 and tapped on tissue paper.
17. TMB was prepared by adding equal volumes of TMB Concentrate to TMB Diluent.
18. 100 μl of TMB was added to each well.
19. The plate was covered with a plate sealer and incubated at 34° C. Shake Speed 3 for 15 mins.
20. 100 μl of 0.5M H₂SO₄ Stop Solution was added to each well to stop the reaction.
21. The plate was read at 450-690 nm and the results saved. The settings for the Tecan sunrise reader settings were: absorbance, 450 nm-690 nm, shake low intensity outside 10 sec. no settle time.

### Buffer Preparation

### Sodium Hydrogen Carbonate Solution

| | | |
|---|---|---|
| Sodium Hydrogen | 4.2 | g |
| Carbonate (NaHCO₃) | | |
| Deionised Water | 1000 | ml |

### Sodium Carbonate Solution

| | | |
|---|---|---|
| Sodium Carbonate (Na₂CO₃) | 5.2 | g |
| Deionised Water | 1000 | ml |

### Coating Buffer

| | |
|---|---|
| Sodium Hydrogen | 1000 ml |
| Carbonate (NaHCO₃) | |
| Solution | |
| Sodium Carbonate | until pH 9.5 is achieved |
| Solution (Na₂CO₃) | |

| | |
|---|---|
| PBS Tablet | 1 tablet |
| Deionised Water | 200 ml |

### Blocking Buffer

| | |
|---|---|
| PBS | 1000 ml |
| DGS (1 + 10) | 100 ml |

### Sample Dilution Buffer

| | |
|---|---|
| 1% Casein | 720 ml (made in PBS) |
| DGS (1 + 10) | 72 ml |

### Conjugate Dilution Buffer

| | |
|---|---|
| EW2 (1X) | 100 ml |
| DGS (1 + 50) | 2 ml |

### Results

The inventors grouped the antigens into three different categories and each category has been specified with unique strains differentiation or early bovine TB detection ability (FIG. 1). The 1^{st} group comprises RD3 deletion (Rv1573) which can differentiate between M. bovis wild type and bovine PPD. The 2^{nd} group comprises early antigens (Rv3616c, ESAT6, CFP10 & α-crystallin 2) which have the ability to detect early bovine TB infection. The 3^{rd} group (MPB70, MPB83 & PE35) comprises antigens covering the complete life-cycle of M. bovis wild type.

### Expression and Purification of Proteins

Transformed cells harbouring the correct constructs for target proteins (the sequences of which are shown in FIG. 2(a)) were selected by colony PCR and sequencing. Expressed target proteins were identified by SDS PAGE analysis of cell lysates (Data not shown). Each target protein was successfully purified and polished by IMAC following IPTG induced expression and endotoxin removal resins respectively. Protein purity & endotoxin level (Quality Control) was confirmed using SDS-PAGE Commassie blue stain, Sliver stain, Western blot analysis (E.C.L./DAB) and endochrome kit.

### ELISA Assays

### Sensitivity Summary

| | | | Samples | |
|---|---|---|---|---|
| Samples | No. | Proteins used | detected | % |
| Study 1 | 291 | MPB70 only | 203/291 | 69.7 |
| Study 2 | 114 | 7 Proteins | 94/114 | 82.5 |
| Study 3 | 61 | 8 Proteins | 59/61 | 96.7 |

7 Proteins Panel = MPB70, MPB83, Rv3616c, ESAT6, CFP10, a-crystallin2, PE35
8 Proteins Panel = MPB70, MPB83, Rv3616c, ESAT6, CFP10, a-crystallin2, PE35 and PPE68.

In addition, the samples employed in study 3 were analysed using only MPB83, ESAT-6 and CFP-10 antigens only and compared with the results with the same cohort of animals as tested with the eight protein panel as described above. It was found that, using MPB83, ESAT-6 and CFP-10 only, the maximum sensitivity obtained in identifying tuberculosis was 86.9%. As noted above, with all eight antigens, the sensitivity was increased to 96.7%, representing a highly significant improvement in sensitivity when employing the panel of the eight specific antigens of the invention.

### EXAMPLE 2

### Multiplex Assays

### Methods

### Serum Samples

Serum samples used in the study were obtained from several sources. Blood samples were taken into serum tubes (Vacuette, serum clot activator tubes, Greiner-bio-one), transported at room temperature and then stored at 2-8° C. until processing. Following centrifugation (3000 g, 30 minutes at 2-8° C.) the serum was removed, aliquoted and stored at −20° C.

Two sets of sera were obtained, one from a negative sample bank including sera from herds of animals with a known history of being free of Mbv. A third group of sera were collected from animals that were proven to be positive for Mbv infection at the time of slaughter based on subsequent histopathological/bacteriological examination.

The fourth set of serum samples was part of an infectivity study. The animals were non-vaccinated and challenged via the intra-tracheal route with a low dose of a virulent strain of Mbv (approximately 5,000 CFU). Sera were collected prior to challenge and then at 2, 5, 10 and 17 weeks post infection (PI) (data not shown). A single intradermal comparative cervical tuberculin test (SICCT) was carried out prior to challenge and also during week 15 PI. All animals in the study had lesions typical of a TB infection, and all animals at 17 weeks PI were culture positive for Mbv.

### Preparation of Antigens

The genes encoding different TB proteins were expressed in E. coli as N-terminal polyhistidine-tagged (6×HIS) fusion proteins by Fusion Antibodies Ltd. (Belfast) using the Fusion Expression Technology (FET) platform. Recombinant proteins were purified and polished to near homogeneity by using Fusion Antibodies Ltd. three-step chromatographic protocol. SDS-PAGE and western blot analysis were performed on all purified and polished recombinant TB proteins to confirm their level of purity was greater than 99%. Synthetic peptides were synthesised by Genosphere Biotechnologies (France) to a purity of >80% (RP-HPLC at 220 nm). Amino acid residues were added to peptides (see FIG. 2(b)(ii) to enhance the hydrophilicity of the peptides. Lyophilised peptides were re-constituted to 2 mg/ml in sterile phosphate buffer saline pH 7.4 (Sigma Aldrich, Dublin) and aliquots were prepared and stored at −20° C. Antigen quantification was performed using the micro BCA™ protein assay kit (Pierce, Rockford, Ill.). See FIGS. 2b(i) and 2b(ii) for the list of antigens (recombinant proteins and synthetic peptides) used in this study.

### Multiplex Antigen Printing

Antigens used in the study were printed in each well of a black polystyrene 96 well plate in a multiplex planar array format by Quansys Biosciences, (Logan, Utah). Optimization of the antigen printing was carried out in conjunction with Enfer. Plates were printed by Quansys Biosciences, (Logan, Utah) and testing of plates was carried out at Enfer Scientific (Naas, Ireland). Buffer mediated alterations in spot deposition and morphology were examined at Quansys Biosciences using an Alpha Innotech 8900 imager workstation and a Nikon Diaphot ELWD phase contrast inverted microscope. The effects of differing buffers used for spotting were screened for differential responses at Enfer. The buffer selected gave the optimal pixel intensity response with the optimal spot size/morphology on the plates. Antigen coating concentrations optimization was carried out at Enfer Scientific (Naas, Ireland) using a series of coating titrations printed by Quansys Biosciences in the optimized printing buffer. Panels of antigens were printed using the optimized printing protocol and buffers and were used for sample testing. All plates were shipped and stored at 2-8° C. Plates were allowed to warm to room temperature for 30 minutes prior to use.

### Multiplex Immunoassay Method

The assay method was developed in-house at Enfer Scientific (Naas, Ireland). Serum samples were diluted 1:250 into sample dilution buffer (Enfer Buffer A, Enfer Scientific, Naas, Ireland) and mixed. 30 μl of sample was added per well. Sample dilution and plating was carried out using the automated pipettor Tecan Genesis RSP 150. The plates were incubated at room temperature with agitation (900 rpm) for 30 minutes. The plates were washed with 1× Enfer Wash Buffer (Enfer Scientific, Ireland) six times and aspirated. The detection antibody (polyclonal rabbit anti-bovine immunoglobulin —HRP, Dako, Denmark) was prepared to a dilution of 1:3000 in detection antibody dilution buffer (Enfer Buffer B, Enfer Scientific, Naas, Ireland). After addition of 30 μl of the detection antibody to test wells, the plates were incubated at room temperature for 15 minutes with agitation (900 rpm). The plates were washed as above and 30 μl of substrate (50:50 dilution of substrate and diluent) added per well (Chemilumenscent substrate and diluent, Quansys Biosciences, Logan, Utah). Signals were captured over a 45 second exposure time using Quansys Biosciences Imaging system (Quansys Biosciences, Logan, Utah). Images were saved as CR2 image files. Data were extracted from the captured images using the Quansys Q-View Software™ Version 2.0. The data were compiled and analysed in a custom made macro in Microsoft Excel (Enfer Multiplex Macro, Version 1.0.1.0).

### Anigen Lateral Flow Testing of Sera

A selection of serum samples were tested according to the manufacturers instructions using the lateral flow test kit from Anigen (Korea). Following warming to room temperature, 4 drops of the sera were added to the sample deposition area on the lateral flow cassette. The test was read after 20 minutes. Only tests with a positive control line were considered for further evaluation.

### Results

### Development of the Multiplex Chemiluminescent ELISA

Preliminary studies were conducted to optimize and obtain uniform binding of each antigen using a fluorescent tag. Following optimization of spotting, tests were performed to determine lot to lot variability in pixel intensity at Quansys Biosciences based on the fluorescent tag incorporated into the printing buffer. Plates were tested and evaluated by Enfer for signal variation using a panel of serum samples. Testing of the plates with the detection antibody, with and without sera from a panel of control sera from uninfected cattle, showed that background noise in the system in areas of the surface not coated with antigens was on an average 779.3 relative light units (RLU). Optimisation of the printing concentration for all antigens was carried out on a series of plates printed based on a cohort of control sera. Optimal printing concentrations were determined and used in subsequent testing. Buffers, blocking agents and assay conditions were further optimised on the multiplex assay as well as optimisation of the exposure time for signal capture. The optimal exposure time was determined to be 45 seconds, this giving the best signal to noise ratios with a cohort of samples with varying signal intensities (data not shown).

A total of 1489 TB negative and 522 TB positive animal sera were screened against the antigens listed in FIGS. 2b(i) and 2b(ii). The individual specificity and sensitivity of all antigens were calculated based on a range of cut offs before selecting individual thresholds for each individual antigen. The individual sensitivities of the antigens ranged from 5.4-95.0% while individual specificities ranged from 69.1-99.1%. Results for the overall sensitivity and specificity of the assay are shown in table 3. Calculation of results was based on a combinational approach to the data analysis on results from all antigens for each sample. The individual specificities and sensitivities for ESAT-6 and CFP-10 antigens are shown for these test groups (Table 3).

Table 3 Cohorts of samples tested by Enfer Multiplex assay platform, the Anigen lateral flow kit and figures for individual responses on ESAT-6 and CFP-10 recombinant antigens (data extracted from the multiplex assay for these individual antigens) shown here.

TABLE 3

| | n, TB | | n, TB | |
|---|---|---|---|---|
| | Positive | Sensitivity | negative | Specificity |
| Test | animals | (%) | animals | (%) |
| Enfer | 522 | 93.1 | 1489 | 98.4 |
| Multiplex | | | | |
| assay | | | | |
| ESAT-6 | 522 | 40.6 | 1489 | 86.6 |
| CFP-10 | 522 | 82.6 | 1489 | 69.7 |
| Anigen | 214 | 83.6 | 79 | 83.0* |
| Lateral | | | | |
| Flow | | | | |

*reported accuracy of the Anigen lateral flow kit is 85.5%

### Screening of Infectivity Study Samples

A total of 5 animals that were challenged with a dose of approximately 5,000 CFU Mbv were tested on the Enfer multiplex assay as described in the methods above. Serum samples were tested from the animals at the following time points, pre-challenge time zero and then 2, 5, 10 and 17 weeks post challenge. All animals were determined as having been positively infected by 10 weeks post infection.

Three of the 5 animals were detected at 5 weeks. Table 4 shows the results for this series of samples from the animals over the course of the infectivity study. The time points at which each animal was determined as being positive on the Enfer multiplex ELISA, as well as results for ESAT-6 and CFP-10 antigens are shown.

TABLE 4
Serum samples from infectivity study (A-E)
at times points: pre-challenge, 2, 5, 10 and 17
weeks post challenge. Sample grading is based on
the number of antigens used in the Enfer multiplex
immunoassay giving a positive response. The
individual positive responses for ESAT-6 and CFP-10
antigens (data extracted from the multiplex assay
for these individual antigens) are also shown for
comparison.

| | | Enfer | | |
|---|---|---|---|---|
| | week | Multiplex | | CFP- |
| Sample | number | immunoassay | ESAT-6 | 10 |
| A | 0 | − | − | − |
| A | 2 | − | − | − |
| A | 5 | ++ | − | − |
| A | 10 | ++ | − | − |
| A | 17 | +++ | − | − |
| B | 0 | − | − | − |
| B | 2 | + | − | + |
| B | 5 | − | − | − |
| B | 10 | + | − | − |
| B | 17 | +++ | − | + |
| C | 0 | − | − | − |
| C | 2 | − | − | − |
| C | 5 | − | − | − |
| C | 10 | + | + | − |
| C | 17 | ++++ | + | + |
| D | 0 | − | − | − |
| D | 2 | − | − | − |
| D | 5 | ++ | − | − |
| D | 10 | +++ | − | − |
| D | 17 | ++ | − | − |
| E | 0 | − | − | − |
| E | 2 | − | − | − |
| E | 5 | ++ | − | − |
| E | 10 | ++ | − | + |
| E | 17 | +++ | − | − |

FIG. 3 shows an example of the captured image of the test wells from the multiplex ELISA for one of the animals from the infectivity study. FIG. 4 illustrates Image of 96 well plate from the multiplex assay. Negative and positive controls are located in wells 1 A-E. This plate shows a representative group of TB positive and negative animal samples screened against 11 antigens.

### Comparison of Anti-Mortem Tests to Post Mortem Tests

The results for the selection of samples tested on the Anigen lateral flow cassette were compiled alongside the data from the Enfer Multiplex Immunoassay and histopathological/bacteriological examination results. To calculate the sensitivity of the tests a maximum of 522 animals were considered that had confirmed histopathological/bacteriological data. Analysis of results from the Enfer Multiplex Immunoassay had a specificity of 98.4% (1489 confirmed negative animals), with a sensitivity of 93.1% for the sera tested. When the same comparison was made for the Anigen lateral flow kit, which has a reported accuracy of greater or equal to 85.5%, a tested specificity of 84.2% (79 negative animal samples tested) and a sensitivity of 84% was observed with the kit (214 positive animals tested). The Anigen lateral flow test kit uses a recombinant MPB70 antigen for detection of antibodies.

### EXAMPLE 3

### Identification of Panel of Antigens for Diagnosis of TB in Bovines

The inventors used the multiplex platform described above to analyse antigen expression from 522 cattle, which had been confirmed TB positive by histopathology and culture methods, using 1489 cattle, which had been confirmed TB negative using tuberculin skin tests, as the controls. The inventors identified a panel of five antigens which when assayed together enable diagnosis of tuberculosis infection with very high sensitivity and selectivity. The assaying for the presence of at least two of the five antigens ESAT-6+Rv3616c+pep1 (MPB70)+MPB83+MPB70 gave a sensitivity of 93.7%, compared to 71.4% obtained with ESAT-6+Rv3616c+pep1 (MPB70)+MPB83, 25.5% obtained using ESAT-6+Rv3616c+pep1 (MPB70). The specificity obtained with ESAT-6+Rv3616c+pep1 (MPB70)+MPB83+MPB70 was 97.6%.

### EXAMPLE 4

### Identification of Panel of Antigens for Diagnosis of TB in Badgers

The inventors used the multiplex platform described above to analyse antigen expression from 67 badgers, which had been confirmed TB positive by histopathology and culture methods, using 133 badgers, which had been confirmed TB negative using tuberculin skin tests, as the controls. The inventors identified a panel of three antigens which when assayed together enable diagnosis of tuberculosis infection with sensitivity and selectivity at levels hitherto not obtained with badgers. The assaying for the presence of at least one of the three antigens Rv3616c+MPB83+MPB70 gave a sensitivity of 58.2%, compared to 52.2% obtained with Rv3616c+MPB70 alone, and 31.3% obtained using MPB70 alone. The specificity obtained with Rv3616c+MPB83+MPB70 was 96.2%.

The inventors have developed a robust rapid diagnostic assay for Mbv infection using a multiplex system that can simultaneously detect and analyze antibody activity to multiple antigens. For example, up to 25 antigens can be spotted in a grid format in a single well in a 96 well format. The format can be configured for a larger array of antigens if needed. The assay is rapid and is designed for use with a single plate or use in an automated system with the potential for high throughput. Importantly, software has been developed for image capture and rapid analysis of large numbers of serum samples.

Attempts in the past to develop a robust assay system based on the bovine humoral immune response to bTB have been unsuccessful due to a lack of sensitivity and specificity of the assays. This is in part attributable to variations in the antibody response to Mbv antigens at different stages of infection. The use of a multiplex assay that contains a combination of Mbv specific proteins representative of different stages of disease optimizes the ability to diagnose of this complex disease As reported here the inventors have succeeded in identifying a combination of immune dominant proteins capable of providing a rapid and accurate diagnosis of bTB (bovine TB) status. The inventors have obtained a sensitivity of 93% and specificity of 98% with banks of known negative and positive sera. The sensitivity and specificity was higher than that obtained with a commercial lateral flow assay and an ELISA. They were able to detect antibody activity at dilutions up to 1/250, with the optimized coating concentrations for the antigens and the optimized assay reagents and multiplex platform.

Due to the inclusion of 20 antigens of importance in this multiplex immunoassay the testing of other species is possible on the same platform. Indeed, the inventors have identified particular combinations of antigens which may be used to diagnose the presence of TB in badgers.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. An assay for the detection of the presence of Mycobacterium, for example Mycobacterium bovis, in a biological sample, said method comprising the steps:
providing a biological sample from said animal; and determining the presence or absence of a group of antigens, or the presence or absence of an immune response to each member of said group of antigens, wherein said group of antigens comprises:
(i) the group consisting of an Rv3616 antigen, an MPB70 antigen, an MPB83 antigen, and an ESAT 6 antigen; or
(ii) at least seven of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen;
in the biological sample;
wherein the identification of the presence of two or more of said antigens or of an immune response thereto is indicative of the presence of Mycobacterium in the biological sample.

2. The method according to claim 1, wherein the Mycobacterium is Mycobacterium bovis.

3. A method of diagnosis of tuberculosis in an animal, the method comprising the steps:
providing a biological sample from said animal; and determining the presence or absence of, or an immune response to, a group of antigens, wherein said group of antigens comprises:
(i) the group consisting of an Rv3616 antigen, an MPB70 antigen, an MPB83 antigen, and an ESAT 6 antigen; or
(ii) at least seven of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen;
wherein the identification of the presence of two or more of said antigens of said group of antigens, or of an immune response thereto, is indicative of the presence of tuberculosis infection in the animal.

4. The method according to claim 1 comprising determining the presence or absence of each of the antigens, or an immune response to each of the antigens, of the group consisting of an Rv3616 antigen, an MPB70 antigen, and MPB83 antigen, an ESAT 6 antigen, a CFP 10 antigen, an α crystalline 2 antigen, a PE35 antigen, and a PPE68 antigen, in the biological sample.

5. The method according to claim 1, wherein the method comprises determining the presence or absence of a group of antigens, or the presence or absence of an immune response to each member of said group of antigens, wherein said group of antigens comprises both a full length MPB70 protein and an MPB70 linear epitope, for example PEP1 (MPB70).

6. The method according to claim 1, further comprising determining the presence or absence of at least one of the antigens, or an immune response to at least one of the antigens, of the group consisting of an Rv1573 antigen, an Rv1580c antigen or an Rv1585c antigen.

7. The method according to claim 6, wherein the identification of the presence of one or more of the antigens, or an immune response to at least one of the antigens, of the group consisting of an Rv1573 antigen, an Rv1580c antigen or an Rv1585c antigen is indicative of the presence of, or infection with, Mycobacterium bovis.

8. A method of determining whether an animal is infected with tuberculosis or vaccinated against tuberculosis, said method comprising the steps:
(a) providing a biological sample from said animal; and
(b) determining the presence or absence in the biological sample of:
(i) at least three of the group consisting of an Rv3616 antigen, an MPB70 antigen, and MPB83 antigen, an α crystalline 2 antigen, or an immune response thereto; and
(ii) two or more of the group consisting of an ESAT6 antigen, a CFP10 antigen, a PE35 antigen, and a PPE68 antigen or an immune response thereto;
wherein the identification of the presence of (i) at least one of the group consisting of an Rv3616 antigen, an MPB70 antigen, and MPB83 antigen, and an α crystalline 2 antigen, or an immune response thereto, and (ii) at least one of the group consisting of an ESAT6 antigen, a CFP10 antigen, a PE35 antigen, and a PPE68 antigen, or an immune response thereto is indicative of the presence of infection of the animal with tuberculosis; and
wherein the identification of the presence of at least one of the group consisting of an Rv3616 antigen, an MPB70 antigen, and MPB83 antigen, and an α crystalline 2 antigen, or an immune response thereto, combined with the absence of each of the antigens tested of (ii), or an immune response thereto, is indicative of vaccination of the animal with a vaccine.

9. The method according to claim 8, wherein step (b) further comprises determining the presence or absence in the biological sample of:
(iii) at least one of the group consisting of an Rv1573 antigen, an Rv1580c antigen or an Rv1585c antigen, or an immune response thereto;
wherein the determination of the absence of each of the antigens tested of (iii), or an immune response thereto, in combination with the determination of the absence of each of the antigens tested of (iii), or an immune response thereto, and the identification of the presence of at least one of the group consisting of an Rv3616 antigen, an MPB70 antigen, and MPB83 antigen, and an α crystalline 2 antigen, or an immune response thereto is indicative of vaccination of the animal with a vaccine.

10. The method according to claim 9, wherein the vaccine is a BCG vaccine.

11. A method of diagnosis of tuberculosis in a mammal, for example a reservoir mammal, the method comprising the steps:
providing a biological sample from said animal; and determining the presence or absence of, or an immune response to an Rv3616 antigen, an MPB70 antigen, and an MPB83 antigen, wherein the identification of the presence of one or more of said antigens of said group of antigens, or of an immune response thereto, is indicative of the presence of tuberculosis infection in the mammal.

12. The method according to claim 1 wherein the presence of said one or more antigens is determined by determining the presence of an antibody response to said antigens.

13. The method according to claim 1 wherein the presence of said one or more antigens is determined by determining the presence of a nucleic acid encoding said polypeptide or by determining the presence of a nucleic acid encoding an antibody to said polypeptide.

14. A diagnostic kit for the diagnosis of the presence of tuberculosis in a subject, said kit comprising:
at least seven of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

15. The kit according to claim 14, wherein the kit comprises each of the antigens of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

16. A diagnostic kit for the diagnosis of the presence of tuberculosis in a subject, said kit comprising an Rv3616 antigen, an MPB70 antigen, an MPB83 antigen, and an ESAT 6 antigen.

17. The diagnostic kit according to claim 16, wherein the kit comprises both an MPB70 antigen which is a full length MPB70 protein and an MPB70 antigen which is an MPB70 linear epitope, for example PEP1 (MPB70).

18. The kit according to claim 14, further comprising at least one of the antigens selected from the group consisting of an Rv1573 antigen, an Rv1580c antigen or an Rv1585c antigen.

19. A diagnostic kit for the diagnosis of the presence of tuberculosis in a biological sample, said kit comprising:
at least seven of the group consisting of an antibody molecule with binding specificity for an Rv3616 antigen, an antibody molecule with binding specificity for an MPB70 antigen, an antibody molecule with binding specificity for an MPB83 antigen, an antibody molecule with binding specificity for an ESAT 6 antigen, an antibody molecule with binding specificity for a CFP10 antigen, an antibody molecule with binding specificity for an α crystalline 2 antigen, an antibody molecule with binding specificity for a PE35 antigen, and an antibody molecule with binding specificity for a PPE68 antigen.

20. The kit according to claim 19, wherein the kit comprises each of an antibody molecule with binding specificity for an Rv3616 antigen, an antibody molecule with binding specificity for an MPB70 antigen, an antibody molecule with binding specificity for an MPB83 antigen, an antibody molecule with binding specificity for an ESAT 6 antigen, an antibody molecule with binding specificity for a CFP10 antigen, an antibody molecule with binding specificity for an α crystalline 2 antigen, an antibody molecule with binding specificity for a PE35 antigen, and an antibody molecule with binding specificity for a PPE68 antigen.

21. A diagnostic kit for the diagnosis of the presence of tuberculosis in a biological sample, said kit comprising:
an antibody molecule with binding specificity for an Rv3616 antigen, an antibody molecule with binding specificity for an MPB70 antigen, an antibody molecule with binding specificity for an MPB83 antigen.

22. The diagnostic kit according to claim 21, wherein the kit comprises both an antibody molecule with binding specificity for full length MPB70 protein and an antibody molecule with binding specificity for an MPB70 linear epitope.

23. The kit according to claim 19, further comprising at least one of the antibody molecules selected from the group consisting of an antibody molecule with binding specificity for an Rv1573 antigen, an antibody molecule with binding specificity for an Rv1580c antigen, and an antibody molecule with binding specificity for an Rv1585c antigen.

24. A diagnostic kit for the diagnosis of the presence of tuberculosis in a reservoir animal, said kit comprising an Rv3616 antigen, an MPB70 antigen, and an MPB83 antigen.

25. A diagnostic kit for the diagnosis of the presence of tuberculosis in a biological sample from a reservoir animal, said kit comprising: an antibody molecule with binding specificity for an Rv3616 antigen, an antibody molecule with binding specificity for an MPB70 antigen, and an antibody molecule with binding specificity for an MPB83 antigen.

26. A vaccine comprising at least seven of the antigens, or variants or fragments thereof, of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

27. The vaccine according to claim 26, where the vaccine comprises an antigen or fragment or variant thereof of each member of the group consisting of an Rv3616 antigen, an MPB70 antigen, and MPB83 antigen, an ESAT 6 antigen, a CFP10 antigen, an α crystalline 2 antigen, a PE35 antigen, and a PPE68 antigen.

28. A vaccine for reservoir animals, said vaccine comprising an Rv3616 antigen or variant or fragment thereof, an MPB70 antigen or variant or fragment thereof, and an MPB83 antigen or variant or fragment thereof.
